(19) United States
(12) Patent Application Publication
(10) Pub. No.: US 2022/0101945 A1
(43) Pub. Date: Mar. 31, 2022

(54) SPECIFIC STRUCTURAL VARIANTS DISCOVERED WITH NON-MENDELIAN INHERITANCE

(71) Applicant: UT-Battelle, LLC, Oak Ridge, TN (US)

(72) Inventors: Michael R. Garvin, Oak Ridge, TN (US); Daniel A. Jacobson, Oak Ridge, TN (US); David Kainer, Oak Ridge, TN (US); Erica Teixeira Prates, Oak Ridge, TN (US)

(21) Appl. No.: 17/487,188

(22) Filed: Sep. 28, 2021

Related U.S. Application Data
(60) Provisional application No. 63/084,151, filed on Sep. 28, 2020.

(51) Int. Cl. G16B 20/20; G16B 30/00; G16B 40/00

(52) U.S. Cl. CPC G16B 20/20; G16B 40/00; G16B 30/00

(57) ABSTRACT

The present disclosure is directed to methods of identifying structural variants (SVs) from single nucleotide polymorphisms (SNPs) that demonstrate non-Mendelian inheritance pattern (NMI) and finding the biological relevance of the SVs through machine learning. Also disclosed are processors programmed to identify biologically-relevant SVs and computer-readable storage devices comprising instructions to identify biologically-relevant SVs.

### CROSS REFERENCE TO RELATED APPLICATION

This application claims the benefit of priority from U.S. Provisional Application No. 63/084,151, filed Sep. 28, 2020, the entire contents of which are incorporated herein by reference.

### STATEMENT REGARDING FEDERALLY SPONSORED RESEARCH OR DEVELOPMENT

This invention was made with government support under contract no. DE-AC05-00OR22725, awarded by the United States Department of Energy. The government has certain rights in the invention.

### BACKGROUND

Structural variants (SVs) are genomic changes that include deletions, insertions, and inversions which have much greater effects on an individual phenotype than single nucleotide polymorphism (SNPs). SVs are fifty times more likely to affect the expression of a gene, and three times more likely to be associated with a positive signal from a genome wide association study (GWAS) compared to a SNP. It is now widely accepted that SVs are likely responsible for many diseases and disorders, but detecting them with short-read sequencing (e.g., Illumina next-generation sequencing) is difficult and these approaches are only capturing about 40% of the true SVs that exist in the human population. Furthermore, that estimate is an average over all types of SVs and for specific types, such as mobile element insertions, they are likely only capturing 5-10%. Finally, despite the fact that identifying SVs with short-read sequencing fails to find most existing SVs, it requires substantial effort, multiple algorithms, and an accurate reference genome. As a consequence, SV detection in non-human species will be even more difficult, yet no less important from the perspective of agriculture, forestry and ecology. What is needed is an in expensive and rapid method to accurately detect SVs in any species on a population scale.

### SUMMARY OF THE DISCLOSURE

An aspect of this disclosure is directed to a method of identifying at least one structural variation in a genome, the method comprising: assembling single nucleotide polymorphism (SNP) data from parents and their offspring; analyzing the SNP data for a plurality of non-Mendelian inheritance (NMI) patterns, wherein each NMI in the plurality of NMI patterns is a potential structural variation (SV); scoring the NMIs to identify large structural variations, wherein a run of at least three SNPs with NMI indicates a large structural variation; removing SNPs that demonstrate NMI in the offspring but that overlap with at least one known existing variation; identifying conserved regions of the genome to filter regions that should be conserved but include a structural variation; identifying biologically important structural variations; and classifying the identified biologically important structural variations using a machine learning algorithm.

In some embodiments, the genes in which the identified SVs reside points to treatments based on known mechanisms of action of the gene. For instance, an SV in an NMDA receptor may indicate that the subject would respond to NMDA agonists or antagonists. Each individual's list of SVs based on NMI can be used to tailor a personalized treatment plant for that individual.

In some embodiments, the machine learning algorithm is a neural network.

In some embodiments, the machine learning algorithm is an iterative Random Forest.

In some embodiments, the method further comprises determining the frequency an NMI and comparing the frequency of NMIs at the corresponding genomic location in a population, and determining that the NMI indicates a structural variation if the frequency of the NMI is higher than that of the corresponding genomic region in the population.

In some embodiments, the biologically important structural variations are selected from structural variations that reside in a gene in which less than 5% of normal individuals have a known structural variation; and there is a run of at least four SNPs with NMI in a row.

In some embodiments, identifying conserved regions of the genome is performed by a custom correlation coefficient (CCC) analysis.

In some embodiments, the method further comprises assigning a probability on having a run of NMI and maintaining SNP's with a run of NMI greater than 4.

In some embodiments, the method further comprises removing NMI attributable to high levels of masked repetitive elements.

In some embodiments, the method further comprises identifying pinpoint locations of the structural variations and identifying pinpoint locations of conserved blocs of genetic information.

In some embodiments, the method further comprises using the locations of the structural variations and the locations of the conserved blocs of genetic information to identify locations of rare structural variations in genes that have conserved blocs of genetic information.

Another aspect of this disclosure is directed to a computer-implemented method of training a machine learning algorithm for identifying at least one structural variation in a genome, the method comprising training the machine learning algorithm using a training set, wherein the training set is created by: assembling single nucleotide polymorphism (SNP) data from parents and their offspring; analyzing the SNP data for a plurality of non-Mendelian inheritance patterns (NMI), wherein each NMI is a potential structural variation; scoring the NMIs to identify large structural variations, wherein presence of at least three neighboring SNPs that demonstrate NMI in the offspring indicates a large structural variation; removing SNPs that demonstrate NMI in the offspring but that overlap with at least one known existing variation; identifying conserved regions of the genome to filter regions that should be conserved but include a structural variation; and identifying potentially biologically important structural variations.

In some embodiments, the machine learning algorithm is a neural network.

In some embodiments, the machine learning algorithm is an iterative Random Forest.

Another aspect of this disclosure is directed to a processor programmed to perform: assembling single nucleotide polymorphism (SNP) data from parents and their offspring; analyzing the SNP data for a plurality of non-Mendelian inheritance (NMI) patterns, wherein each NMI in the plurality of NMI patterns is a potential structural variation; scoring the NMIs to identify large structural variations, wherein the presence of at least three neighboring SNPs that demonstrate NMI in the offspring indicates a large structural variation; removing SNPs that demonstrate NMI in the offspring but that overlap with at least one known existing variation; identifying conserved regions of the genome to filter regions that should be conserved but include a structural variation; identifying biologically important structural variations; and classifying the identified biologically important structural variations using a machine learning algorithm.

In some embodiments, the machine learning algorithm is a neural network.

In some embodiments, the machine learning algorithm is an iterative Random Forest.

In some embodiments, the processor is further programmed for determining the frequency an NMI and comparing the frequency of NMIs at the corresponding genomic location in a population, and determining that the NMI indicates a structural variation if the frequency of the NMI is higher than that of the corresponding genomic region in the population.

In some embodiments, the biologically important structural variations are selected from structural variations that reside in a gene in which less than 5% of normal individuals have a known structural variation; and there is a run of at least four SNPs with NMI in a row.

In some embodiments, identifying conserved regions of the genome is performed by a custom correlation coefficient (CCC) analysis.

In some embodiments, the processor is further programmed for assigning a probability on having a run of NMI and maintaining SNP's with a run of NMI greater than 4.

In some embodiments, the processor is further programmed for removing NMI attributable to high levels of masked repetitive elements.

In some embodiments, the processor is further programmed for identifying pinpoint locations of the structural variations and identifying pinpoint locations of conserved blocs of genetic information.

In some embodiments, the processor is further programmed for using the locations of the structural variations and the locations of the conserved blocs of genetic information to identify locations of rare structural variations in genes that have conserved blocs of genetic information.

Another aspect of this disclosure is directed to a computer-readable storage device, comprising instructions to perform: assembling single nucleotide polymorphism (SNP) data from parents and their offspring; analyzing the SNP data for a plurality of non-Mendelian inheritance (NMI) patterns, wherein each NMI in the plurality of NMI patterns is a potential structural variation; scoring the NMIs to identify large structural variations, wherein the presence of at least three neighboring SNPs that demonstrate NMI in the offspring indicates a large structural variation; removing SNPs that demonstrate NMI in the offspring but that overlap with at least one known existing variation; identifying conserved regions of the genome to filter regions that should be conserved but include a structural variation; identifying biologically important structural variations; and classifying the identified biologically important structural variations using a machine learning algorithm.

In some embodiments, the machine learning algorithm is a neural network.

In some embodiments, the machine learning algorithm is an iterative Random Forest.

In some embodiments, the computer-readable storage device further comprises instructions for determining the frequency an NMI and comparing the frequency of NMIs at the corresponding genomic location in a population, and determining that the NMI indicates a structural variation if the frequency of the NMI is higher than that of the corresponding genomic region in the population.

In some embodiments, the biologically important structural variations are selected from structural variations that reside in a gene in which less than 5% of normal individuals have a known structural variation; and there is a run of at least four SNPs with NMI in a row.

In some embodiments, identifying conserved regions of the genome is performed by a custom correlation coefficient (CCC) analysis.

In some embodiments, the computer-readable storage device further comprises instructions for assigning a probability on having a run of NMI and maintaining SNP's with a run of NMI greater than 4.

In some embodiments, the computer-readable storage device further comprises instructions for removing NMI attributable to high levels of masked repetitive elements.

In some embodiments, the processor is further programmed for identifying pinpoint locations of the structural variations and identifying pinpoint locations of conserved blocs of genetic information.

In some embodiments, the computer-readable storage device further comprises instructions for using the locations of the structural variations and the locations of the conserved blocs of genetic information to identify locations of rare structural variations in genes that have conserved blocs of genetic information.

Another aspect of this disclosure is directed to a method comprising: obtaining a biological sample from a subject, detecting in the biological sample whether at least one gene or genomic region selected from Table 1 has a structural variation; and determining that the subject is at risk of Autism Spectrum Disorder if the at least one gene or genomic region has a structural variation.

An aspect of this disclosure is directed to a method comprising: obtaining a biological sample from a subject, detecting in the biological sample whether the GRIK2 gene has a structural variation; and determining that the subject is at risk of Autism Spectrum Disorder if the GRIK2 gene has a structural variation.

An aspect of this disclosure is directed to a method comprising: obtaining a biological sample from a subject, detecting in the biological sample whether the ACMSD gene has a structural variation; and determining that the subject is at risk of Autism Spectrum Disorder if the ACMSD gene has a structural variation.

### BRIEF DESCRIPTION OF THE DRAWINGS

FIGS. 1A-1B. Non-Mendelian Inheritance (NMI) to detect normally segregating SVs (A) an NMI signal can occur when an SV exists under the region of DNA that is targeted by the hybridizing probe (red “X”). In this example scenario, the missing signal from one allele coupled with a normal signal from the other allele produces an erroneous genotype (pedigree on the right) that does not conform to mendelian expectation of the trio. (B) For example, array genotyping of the ASD trio children for SNP rs221465 results in failure of the HWE test (left). PLINK mendel reveals many individuals with NMI (center plot, red dots) at this SNP. However, there are further individuals where the inventors “suspected NMI” (center, orange dots). These individuals are from trios where PLINK had no power to detect NMI as all three individuals were genotyped as A/A, but they co-locate with the NMI individuals on the signal intensity plot. The inventors inferred the genotype calls for NMI and suspected NMI cases (right), and now this SNP conforms to HWE (note that point locations between plots vary slightly due to an applied jitter). Indeed, it is already known that this SNP tags a large common deletion in the NRXN3 gene. The allele frequency of the deletion (“-”) in the ASD population after the NMI-based correction (0.34) is highly similar to the frequency in the 1000 Genome population (0.37).

FIG. 2. NMI Workflow. (1) NMI is used to identify potential SVs from parent-child trios, either with PLINK or manually, and those sites are re-genotyped accordingly. See FIG. S1 for more details. (2) A set of filters are then applied, including removing SVs found in non-ASD studies. (3) The remaining SVs are subjected to several validation processes, including detection of known ASD-related SVs, known ASD-susceptibility, and differentially expressed genes from an ASD brain study. (4) Coding genes that harbored ASD-SVs marked by NMI SNPs found at greater than 15% frequency in both study populations were assessed for significant enrichment of GO Biological Process terms, disease ontology terms, and transcription factor binding sites involved in chromatin remodeling. These genes' ASD-SVs were also clustered to define sub-groups of ASD.

FIGS. 3A-3D. (A) NMI patterns identified over 60,000 likely structural variants (NMI-SV) in the smaller MIAMI data set (blue) and the vast majority (90%) were validated in the larger AGPC data set (pink) with a very similar frequency spectrum. Removal of known SVs from non-ASD populations left 48,009 ASD-specific SVs (ASD-SVs), most of which were rare. (B) There is a considerable overlap of the highest frequency ASD-SVs between the two studies (right) indicating a likely core set of SVs underlying ASD. (C) Density distributions of the number of genes with high-frequency ASD-SVs per individual. This was done separately for the AGPC and Miami cohorts. The number of genes harboring ASD-SVs varies per case, potentially determining the spectrum of ASD phenotype. On average, each individual in AGPC had 371 genes harboring high frequency ASD-SVs, while individuals in MIAMI averaged 347 (D) NMI-SVs identify more known ASD genes than is expected by chance in the SFARI and AutDB data sets and in the recently reported differentially expressed genes in post-mortem brain tissue of ASD individuals. P-values are shown above each comparison of expected and observed counts.

FIG. 4. Dendritic morphogenesis and ASD-SV frequency. An overview of genes involved in dendritic morphogenesis that contain ASD-SVs. The mean frequency of each ASD-SV for the two ASD studies is provided for each gene. The formation of dendritic spines (lower blue-shaded processes) involves proteins of diverse functions that generate synapses with axons (upper gray-shaded process), many of which our method indicates are disrupted by SVs in individuals with ASD. The most numerous are those that directly manipulate the actin cytoskeleton to form the spine (N=97 genes). GRM5, NMDA, and AMPA receptors mediate calcium release. The glutamate signaling pathway is activated by Wnt/β-catenin signaling (green ovals) via TCF4 and the H3K9me3 lysine demethylase KDM4B and is repressed by ARID1B. This effectively links dendritic spinogenesis, glutamate signaling and synaptic organization identified in the GO enrichment analysis as well as chromatin modification identified by using the overlap with the ENCODE database. Many of the most frequently affected glutamate receptor subunits are involved in the early development of the cerebellum.

FIG. 5. ASD-SV frequency in genes that participate in axon guidance. Successful completion of long-distance axonal migration during brain development requires cells at choice points to secrete cues that are recognized by their cognizant receptors on the cone of the axon. The largest number of receptors disrupted by ASD-SV are the ephrins, which are important for the formation of the Superior Colliculus in the tectum portion of the brain. ADAM-type metalloproteinases degrade sensory receptors that are no longer needed so they can be replaced by those required for the next waypoint and are also often disrupted by ASD-SV. The second most frequently disrupted ligand (NTNG1) is associated with the ASD-like Rett Syndrome and Schizophrenia. Several semaphorins (SEMAs) demonstrate ASD-SV as do their cognizant plexin receptors (PLXNs). Mean frequency of ASD-SV for the two ASD studies are provided for each gene.

FIGS. 6A-6C. An ASD-SV impairs glutamate signaling associated with disruption of the GluK2 (encoded by GRIK2) (A) The ASD-SV at SNP rs2051449 is predicted to disrupt a known splice site adjacent to exon 12 bound by PCBP2, SRSF9, and NHRNKP, as identified from the ENCODE project. A recent analysis of SVs identified a 29-base pair insertion at a CCTTₙ repeat near this site. The portion of the protein encoded by exon 12 is important for glutamate binding. Each subunit of the tetrameric GluK2 is composed of an amino-terminal domain (ATD), a ligand binding domain (LBD) and a transmembrane domain. The subunits are distinguished by color (orange, green, red, and blue) and the amino acid region coded by exon 12 is illustrated in one subunit, in grey (left structure). The cryo-EM structure of the complex from Rattus norvegicus, which is 99% identical to the KAR from Homo sapiens, was used here (PDB 5KUF). Main amino acid residues in contact with the glutamate ligand (in yellow, magnified top right) are depicted. T690, E738 and Y764 are absent due to missing exon 12 in GRIK2 (PDB 4UQQ was used to represent the binding site with glutamate). The region encoded by exon 12 interacts with adjacent LBDs (magnified bottom right) and is critical to the functional dynamics of the tetrameric GluK2. (B) Mapping of RNA-seq data from post-mortem brain tissue reveals 10 of 13 ASD individuals display loss of exon 12 whereas only 1 of 10 controls do. (C) Plot of the Illumina array intensity signals for rs2051449 (top) indicates a likely copy number gain at the site. Partitioning of the cohort into those with and without a CNV at rs2051449 identified 12 coding ASD-SVs with significantly differential frequencies (FDR<0.05, DOSV, two in the same gene, PTPRD). Four genes intersected with differentially expressed genes (DEGs) from post-mortem brain tissue from (b). PTPRD and GRIK2 expression levels are significantly correlated in prefrontal cortex from control individuals (0.65, p<0.03) but not those with ASD (−0.08, p<0.79), further supporting the role of the disruption of these genes as a core component of ASD. TPM=transcripts per million.

FIGS. 7A-7B. Association testing of ASD phenotypes using ASD-SV markers. (A) Manhattan plot of association testing of verbal vs. non-verbal phenotype using presence/absence markers of ASD-SVs at 10,108 loci found two significant ASD-SVs after Bonferroni correction (red line). (B) The most significant association resides in a FOS transcription factor binding site that regulates the ACMSD gene, which codes for a key enzyme in the kynurenic acid pathway. Altered levels of quinolinic acid and picolinic acid of this tryptophan catabolic pathway have been associated with several neuropsychiatric disorders including ASD, and a SNP in this gene has been linked to suicidal behavior. The metabolites kynurenic acid and quinolinic acid in this pathway inhibit glutamate signaling via numerous receptor types, one of which (NMDAR) is a therapeutic target for the treatment of ASD.

FIGS. 8A-8B. Identification of ASD subgroups from GWAS. (A) tSNE plot colored according to hierarchical clustering of genic ASD-SVs shows three subgroups of ASD individuals from the AGPC study. (B) ASD clusters can be explained by the most important genes containing ASD-SV according to iterative Random Forest classifiers. The top 10 genes (based on iRF importance score) for a cluster are shown in a heatmap where cells are colored according to the frequency of their resident ASD-SV (blue=low frequency, red=high frequency) and the contrast with the other two clusters is evident in each heatmap. Frequency values are shown in the cells.

FIG. 9. Block diagram of the system in accordance with the aspects of the disclosure. CPU: Central Processing Unit (“processor”).

### DETAILED DESCRIPTION

The present methods use simple patterns of non-Mendelian inheritance (NMI) that are typically used to screen out what is considered to be flawed SNP genotyping assays. A mother with a genotype of A/A at a locus and a father with genotype of G/G should produce all offspring with a genotype of A/G because each child receives one of the two alleles from each of the parental genotypes. However, some offspring are genotyped as A/A, which is incompatible with the law of Mendelian inheritance.

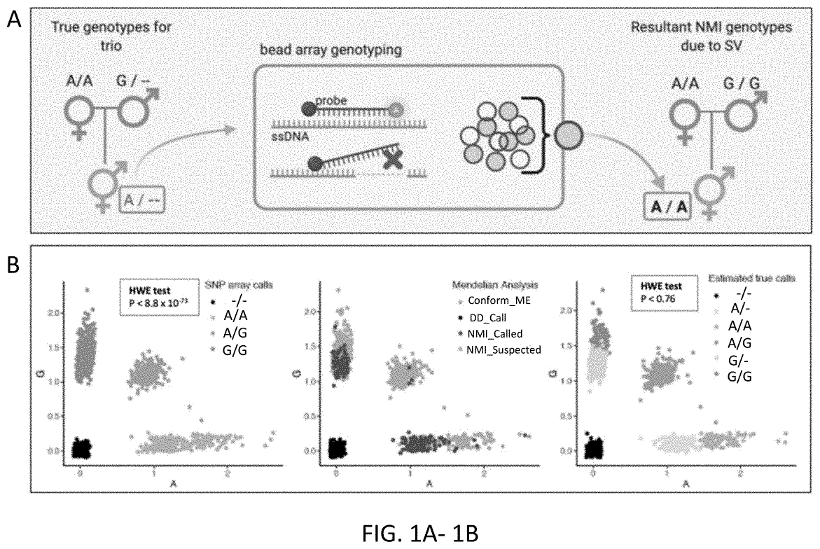

When NMI is used as a filter it is assumed that such loci are due to technical error. However, it is more likely a result of a genotyping assay probe not being able to bind to the region of DNA it is meant to bind to because the sequence targeted by the probe is either mutated or deleted in the individual. This means that only one of the alleles is genotyped (but the assay does not know this), and therefore the offspring appears as a homozygote at this locus but is, in truth, hemizygous for that allele. This is easily seen with large deletions because many adjacent SNPs on the chromosome show the NMI pattern. The inventors then use the detection of NMI as a proxy for the detection of a structural variant. In the case of FIG. 1, there were 43 chromosomally adjacent SNP assays that showed NMI, making it a high confidence SV. The SNP positions on the genotyping array are randomized, so the chance of random genotype probe failure of these 43 SNPs is 8×10⁻¹⁰⁶ based on the overall error rate for the experiment. In addition, when the genotypes are replotter from the raw data and leverage the instant NMI patterns as in FIG. 1B the inventors can identify the true SV genotypes, with high accuracy, that underlie complex disease. For example, these data were generated by SNP genotyping many family trios in which the child has Autism Spectrum Disorder (ASD); there is a known large deletion at the chromosomal region containing the run of 43 adjacent NMI SNPs that has been shown to cause ASD. In FIG. 1C, it is demonstrated that, in this ASD study, after filtering out previously known SVs from studies in non-ASD individuals, 49,464 ASD-specific SVs were detected with the NMI method, most of which were found in coding genes.

Importantly, the inventor further show that these genes are enriched for known ASD-associated genes in (FIG. 1D) and the inventors validate with a truth set of known ASD SVs. From this, the inventors take a Systems Biology approach to uncover the biological meaning and likely functional results of the list of ASD-SVs by layering information from public repositories such as Gene Ontology, Chip-Seq, and PDB. For the GRIK2 gene, the inventors were able to identify the functional implication at the structural level. The inventors also identify specific molecular pathways of dendritic spinogenesis, axon guidance, glutamate signaling, and histone modification that cause the disorder and provide numerous diagnostic and therapeutic targets.

The methods of the instant disclosure have numerous benefits. Currently, the only technology that can efficiently capture SVs missed by short-read sequencing is long-read sequencing, such as PacBio and Oxford Nanopore. However, a drawback to these technologies is that they need significant amounts of high-quality DNA to generate data, and are expensive because one must either sequence at great depth to gain an accurate alignment of a gene of interest, or substantial effort at the lab bench is necessary to target a specific locus or loci of interest because the default mode of these technologies is to sequence the entire genome. The NMI approach is simple and cost effective. SNP genotyping arrays are relatively inexpensive and can target millions of loci at once. In addition, this approach requires that the probe binds on a small region of DNA (typically 50 base-pairs) and, therefore, it does not need the high-quality DNA that long read sequencing technologies do. Finally, there are numerous archived data sets in human and non-human genetic work that can easily be re-analyzed bioinformatically with no laboratory costs.

This application is an improvement over the current field because it uses hierarchical clustering to group the spectrum into subtypes of a disease (e.g., autism, multiple sclerosis) and artificial intelligence to identify the genes that are important to define those subgroups.

The instant methods can be used, for example, for any human genetics and any disease. Numerous personalized medicine companies could implement this approach into their existing data structure immediately and identify thousands of potential therapeutic targets for a myriad of medical conditions. Additionally, agricultural industries for animal and plant products have millions of SNP genotypes on breeding pedigrees and families that could be easily re-mined for SVs linked to valuable traits.

In one embodiment, the disclosure is directed to several potential druggable targets for ASD. The inventors identify ASD-specific SVs in certain subunits of glutamate receptors for which current drug compounds exist and for which others could be developed. One example is the GRIK2 subunit of the kainate-type glutamate receptor. The inventors show that one ASD SV likely removes an exon that encodes part of the binding pocket for the ligand glutamate, so that the protein may still be expressed and assembled in trimers, creating an ineffective receptor. ASD-specific SVs are also common in lysine demethylases, for which many compounds have been developed and tested for the treatment of cancer. These compounds could, for example, be repurposed for tests in ASD or for research in ASD models.

In one embodiment, this method can be used on data from individuals with ASD. In another embodiment, this method can be used on data on any other existing SNP genotype data from families. For example, the method can be used for analyzing data on a set of families with Multiple Sclerosis, and similar analysis can be done on available online data of attention deficit hyperactivity disorder and longevity (human lifespan). In a further embodiment, numerous agricultural products seek to identify genomic features that underlie valuable traits. Future data could be generated with SNP genotyping arrays that are designed to more efficiently capture the NMI signal, e.g., using more SNPs and SNPs with high heterozygosity, which will increase power to detect NMI. Other embodiments include using the instant methods to analyze SNP array data from agricultural and forestry data, where data is often obtained from large numbers of breeding parents and their full-sibling offspring.

Disclosed herein are simple, inexpensive processes for identifying variation in the genome of any sexually reproducing species using non-Mendelian inheritance patterns and the CCC approach from SNP-based genomic data. In some embodiments, the process includes documenting all structural variation (SV) within a single individual. In some embodiments, the SV is tested for association with any trait of interest, including a disease or disorder. In some embodiments, the exact location of the SV is pinpointed and repaired with gene editing technology (such as CRISPR/Cas system, Cre/Lox system, TALEN system and homologous recombination etc.), using the homologous chromosome (the chromosome that does not have the SV) as a guide for repairing the SV. As used herein, the term “CRISPR” refers to a RNA-guided endonuclease comprising a nuclease, such as Cas9, and a guide RNA that directs cleavage of the DNA by hybridizing to a recognition site in the genomic DNA. In some embodiments, somatic cells but not germline cells may be altered, which may limit the effect of the editing to the subject and not affect any future offspring.

Although demonstrated with ASD, the combination of NMI and CCC may be applied to any disorder or disease that has a genetic component. In some implementations, this method may be used to identify any type of SV as small as a few base pairs and as large as several hundred thousand base pairs. In contrast, known methods rely on up to nine computational approaches to map short read technology to a reference (that may contain imputation errors) and then call variants from that mapped reference. In known methods, different approaches are needed to call different types of SV (e.g., deletions vs. inversions) and each layer of statistical inference introduces further bias. Current array-based technology only identifies known SV of relatively large size and of certain types. The methods of the instant disclosure remedy the deficiencies of known methods.

In some embodiments, the SVs identified by the disclosed technology are used to distinguish local populations or ethnic groups and to predict the ancestry of an individual using sequencing data from a biological sample.

In some embodiments, the discovery and identification of SVs with the disclosed technology is used to screen, diagnose, or predict the onset, progression, severity, life expectancy, or general health of an individual.

Various aspects of the present disclosure may be embodied as a program, software, or computer instructions embodied or stored in a computer or machine usable or readable medium, or a group of media which causes the computer or machine to perform the steps of the method when executed on the computer, processor, and/or machine. A program storage device readable by a machine, e.g., a computer readable medium, tangibly embodying a program of instructions executable by the machine to perform various functionalities and methods described in the present disclosure is also provided.

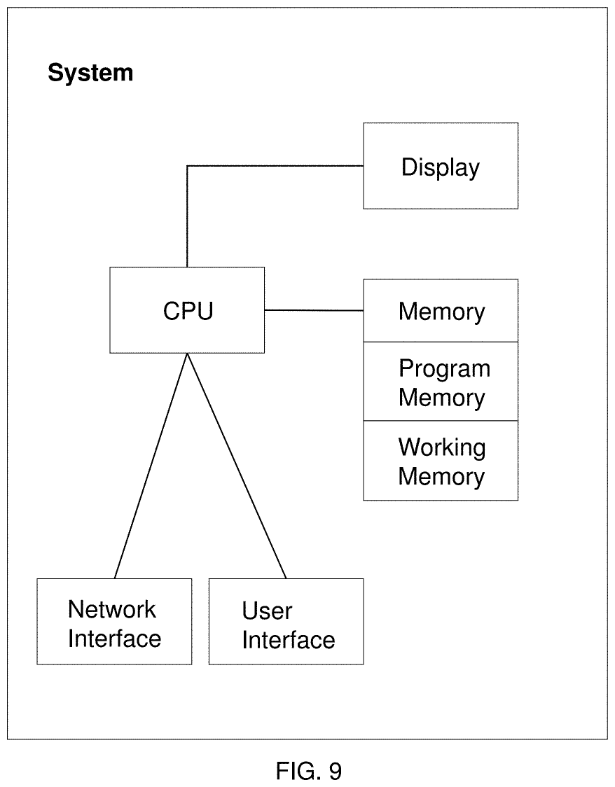

In some embodiments, the present disclosure includes a system comprising a CPU, a display, a network interface, a user interface, a memory, a program memory and a working memory (FIG. 9), where the system is programmed to execute a program, software, or computer instructions directed to methods or processes of the instant disclosure.

### Methods of Identifying Structural Variations

An aspect of this disclosure is directed to a method of identifying at least one structural variation in a genome, the method comprising: assembling single nucleotide polymorphism (SNP) data from parents and their offspring; analyzing the SNP data for a plurality of non-Mendelian inheritance (NMI) patterns, wherein each NMI in the plurality of NMI patterns is a potential structural variation (SV); scoring the NMIs to identify large structural variations, wherein a run of at least three SNPs with NMI indicates a large structural variation; removing SNPs that demonstrate NMI in the offspring but that overlap with at least one known existing variation; identifying conserved regions of the genome to filter regions that should be conserved but include a structural variation; identifying biologically important structural variations; and classifying the identified biologically important structural variations using a machine learning algorithm.

In some embodiments, the genes in which the identified SVs reside points to treatments based on known mechanisms of action of the gene. For instance, an SV in an NMDA receptor may indicate that the subject would respond to NMDA agonists or antagonists. Each individual's list of SVs based on NMI can be used to tailor a personalized treatment plant for that individual.

In some embodiments, the machine learning algorithm is a neural network.

In some embodiments, the machine learning algorithm is an iterative Random Forest.

In some embodiments, the method further comprises determining the frequency an NMI and comparing the frequency of NMIs at the corresponding genomic location in a population, and determining that the NMI indicates a structural variation if the frequency of the NMI is higher than that of the corresponding genomic region in the population.

In some embodiments, the biologically important structural variations are selected from structural variations that reside in a gene in which less than 5% of normal individuals have a known structural variation; and there is a run of at least four SNPs with NMI in a row.

In some embodiments, identifying conserved regions of the genome is performed by a custom correlation coefficient (CCC) analysis.

The CCC algorithm used in this disclosure was developed as a component of the program BlocBuster as described in US 2021/0210162 A1, which is incorporated herein in its entirety. Briefly, this algorithm identifies evolutionary conserved blocs of a genome. The blocs may be regulatory regions that control the expression or splicing of a given gene. Compared to known methods of genetic analysis, the presently disclosed methods, including the combination of CCC and NMI analysis, helps permit accurate identification of CGV.

The CCC program is computationally intensive and can take many computer CPU hours to run. However, the scalability is logarithmic and therefore, reducing the number of SNPs by half decreases processing time by an order of magnitude. This also has the desirable property of removing CCC correlations that are due to physical linkage on a chromosome. To do this, for each CCC analysis, the data is divided into two data subsets to speed processing and to reduce effects of linkage disequilibrium: first, the data is sorted by chromosome and position and then every second SNP was taken for the first data.

In some embodiments, the method further comprises assigning a probability score on having a run of NMI and maintaining SNP's with a run of NMI greater than 4. As used herein, the phrase “a run of NMI” refers to at least three SNPs that are next to each other on a genomic location that show NMI. In some embodiments, a run of NMI greater than 4 represents a large structural variation. In some embodiments, a large structural variation is a deletion of the region of the chromosome. In some embodiments, a run of NMI is greater than 4 SNPs, greater than 5 SNPs, greater than 10 SNPs, greater than 20 SNPs, greater than 30 SNPs, greater than 40 SNPs, or greater than 50 SNPs.

In some embodiments, the method further comprises removing NMI attributable to high levels of masked repetitive elements as described in US 2021/0210162 A1, which is incorporated herein in its entirety. In some embodiments, the presently disclosed methods include additional removal of non-Mendelian hits that could be due to high levels of repetitive elements that are “masked” from downstream analyses, which is a common feature in genomes. Specifically, to determine if a repeat element (such as Short Interspersed Nuclear Elements—SINES—or Long Interspersed Nuclear Elements—LINES) overlapped the NMI and CCC SNPs, the RepeatMasker track in BED format from UCSC Genome Table Browser was uploaded to CLC Genomics. Annotations were overlapped with the SNPs with a range of 50 bp on either side of the SNP of interest that could potentially interfere with the binding of the Illumina probe. The same analysis was performed for all SNPs on the Illumina array to generate an expected frequency for the NMI and CCC data sets. Counts were binned into categories of different transposable elements: ALR/Alpha, Alu (SINES), HERV, LINE1, LINE2, MAM, MIR, THE1, Charlie, HAL, LINE3, LINE4, LTR, MER, MIR, MLTF, and Tigger. A Chi-Square test was done using the frequency from the full Illumina array to generate the expected number of elements in each category for each group (all NMI, NMI with runs greater than 4, and CCC SNPs). A Bonferroni correction (p<0.002) was used to account for multiple tests.

The expectation is that there will be no enrichment for any of the foregoing classes of repetitive elements in genomics regions with SV. If there are enrichments for certain types of repetitive elements in the disease data compared to the data from normal individuals, based on expected frequency (generated from the frequency of each element genome-wide), this may indicate biological relevance. For example, the transposon may be a part of the SV process for a given disease. In the case of Autism, there is an enrichment for active (L1—LINE1) transposable elements and a decrease in the expected number of inactive (L2) elements. L1 transposons are correlated with SV in Autism and may be the underlying cause of the disorder.

In some embodiments, the method further comprises identifying pinpoint locations of the structural variations and identifying pinpoint locations of conserved blocs of genetic information.

In some embodiments, the method further comprises using the locations of the structural variations and the locations of the conserved blocs of genetic information to identify locations of rare structural variations in genes that have conserved blocs of genetic information as determined by a CCC analysis as described herein.

In some embodiments, the genome analyzed by the instant methods is from a subject having or suspected of having a disease. In some implementations, the subject has or suspected of having an autism spectrum disorder (ASD). In some implementation, the subject has or suspected of having multiple sclerosis. In some implementations, the subject has or suspected of having hereditary hemochromatosis.

In some embodiments, the subject is treated with a known intervention, such as a pharmaceutical or non-pharmaceutical approach. Examples of pharmaceutical interventions include small molecules and biologics. Examples of non-pharmaceutical interventions include reducing stimuli (such as reducing noise for a noise-sensitive autistic subject) or physical therapy (such as leg strengthening exercises for a gait-impaired MS subject).

In some implementations, the subject is treated directly or indirectly with a gene editing technology. One example of a gene editing technology is CRISPR. In some implementations, sequence is removed back to the SNPs on either side of the CGV that demonstrate normal Mendelian inheritance. The homologous chromosomal sequence may serve as a guide for with what the SV-altered sequence should be replaced. In some implementations, somatic cells but not germline cells may be altered, which may limit the effect of the editing to the subject and not affect any future offspring. In some implementations, the subject is treated with CAR-T cells. Methods of treating subjects with CAR T cells may follow, for example, the FDA-approved gene therapy methods for tisagenlecleucel (Kymriah®, Novartis, Basel, Switzerland) and/or for axicabtagene ciloleucel (Yescarta®, Gilead, Los Angeles, Calif.). CAR-T cells have been approved for treatment of non-Hodgkin's lymphoma and/or for acute lymphoblastic leukemia, and may be employed to treat other diseases or disorder. In one example, CAR-T cells for the treatment of MS target T cells. In one example, CAR T cells for the treatment of ASD target cells involved in the immune response, such as T cells or cells that secrete inflammatory cytokines such as IL-6 or IL-1β. In one example, CAR-T cells for the treatment of hereditary hemochromatosis target macrophages.

The presently disclosed methods may also be used to identify diagnostic markers, such as networks of genes, for a disease or disorder of interest. The disease or disorder may be any one that has a genetic component. Examples disclosed herein include multiple sclerosis (MS) and autism spectrum disorder (ADS), but the methods are not limited to those diseases and disorders.

### Methods of Training a Machine Learning Algorithm

An aspect of this disclosure is directed to a computer-implemented method of training a machine learning algorithm for identifying at least one structural variation in a genome, the method comprising training the machine learning algorithm using a training set, wherein the training set is created by: assembling single nucleotide polymorphism (SNP) data from parents and their offspring; analyzing the SNP data for a plurality of non-Mendelian inheritance patterns (NMI), wherein each NMI is a potential structural variation; scoring the NMIs to identify large structural variations, wherein presence of at least three neighboring SNPs that demonstrate NMI in the offspring indicates a large structural variation; removing SNPs that demonstrate NMI in the offspring but that overlap with at least one known existing variation; identifying conserved regions of the genome to filter regions that should be conserved but include a structural variation; and identifying potentially biologically important structural variations.

In some embodiments, the machine learning algorithm is a neural network.

In some embodiments, the machine learning algorithm is an iterative Random Forest.

### Processor

An aspect of this disclosure is directed to a processor programmed to perform: assembling single nucleotide polymorphism (SNP) data from parents and their offspring; analyzing the SNP data for a plurality of non-Mendelian inheritance (NMI) patterns, wherein each NMI in the plurality of NMI patterns is a potential structural variation; scoring the NMIs to identify large structural variations, wherein the presence of at least three neighboring SNPs that demonstrate NMI in the offspring indicates a large structural variation; removing SNPs that demonstrate NMI in the offspring but that overlap with at least one known existing variation; identifying conserved regions of the genome to filter regions that should be conserved but include a structural variation; identifying biologically important structural variations; and classifying the identified biologically important structural variations using a machine learning algorithm.

In some embodiments, the processor is part of a system as shown in FIG. 9 comprising a CPU, a network interface, a user interface, a memory and a display.

The term “memory” as used herein comprises program memory and working memory. The program memory may have one or more programs or software modules. The working memory stores data or information used by the CPU in executing the functionality described herein.

The term “processor” may include a single core processor, a multi-core processor, multiple processors located in a single device, or multiple processors in wired or wireless communication with each other and distributed over a network of devices, the Internet, or the cloud. Accordingly, as used herein, functions, features or instructions performed or configured to be performed by a “processor,” may include the performance of the functions, features or instructions by a single core processor, may include performance of the functions, features or instructions collectively or collaboratively by multiple cores of a multi-core processor, or may include performance of the functions, features or instructions collectively or collaboratively by multiple processors, where each processor or core is not required to perform every function, feature or instruction individually. The processor may be a CPU (central processing unit). The processor may comprise other types of processors such as a GPU (graphical processing unit). In other aspects of the disclosure, instead of or in addition to a CPU executing instructions that are programmed in the program memory, the processor may be an ASIC (application-specific integrated circuit), analog circuit or other functional logic, such as a FPGA (field-programmable gate array), PAL (Phase Alternating Line) or PLA (programmable logic array).

The CPU is configured to execute programs (also described herein as modules or instructions) stored in a program memory to perform the functionality described herein. The memory may be, but not limited to, RAM (random access memory), ROM (read-only memory) and persistent storage. The memory is any piece of hardware that is capable of storing information, such as, for example without limitation, data, programs, instructions, program code, and/or other suitable information, either on a temporary basis and/or a permanent basis.

The machine learning algorithm of the instant disclosure improves a computer's ability to analyze and categorize the SVs identified with the NMI analysis described herein. The categorization provided by the instant machine learning algorithm further allows personally tailored treatments based on the genes that are affected by the SVs.

In some embodiments, the machine learning algorithm is a neural network.

In some embodiments, the machine learning algorithm is an iterative Random Forest (iRF). Iterative Random Forest is an improvement over standard Random Forest for datasets with large feature space. It applies feature-selection and boosting to iteratively remove noise and boost true signal. It therefore improves the reliability of the top-ranked (most important) features in the model. In some embodiments, it means that the genes that are determined to be most predictive of each disease cluster are probably more reliable than the equivalent result provided by Random Forest. In some embodiment, the iRF comprises assigning individuals in a single predefined cluster the value of 1, and the rest the value of 0. In some embodiments, the single predefined cluster comprises individuals diagnosed with a particular disease (e.g., ASD, MS etc.) and the rest of the individuals are people not diagnosed with the disease. In some embodiments, the presence/absence for each gene or genomic region is set to 0/1, respectively, and all genes are used as features in the iRF model, which performs an iterative feature selection. In some embodiments, this process is repeated for each of the clusters, resulting in a final random forest for each cluster. In some embodiments, top 10, top 15, top 20, top 25, or top 30 most important genes or genetic regions for each cluster are extracted based on their Gini importance scores provided by the Ranger v0.12 R package.

In some embodiments, clusters of a disease (i.e., groups of cases that are more similar to each other based on which SVs they have) are defined through unsupervised learning algorithms. For a given cluster, all cases in that cluster are given a value of 1, while all ASD cases outside the cluster are given a value of 0. An iRF model is then trained using the SV presence/absence input matrix as features in order to explain the 0 or 1 cluster assignments of the cases. Once the iRF model is fit, the importance score of each input feature (SV) can be obtained so that the SVs can be ranked from most important to least important according to the model.

In some embodiments, there are at least 3 clusters, at least 4 clusters, at least 5 clusters, at least 6 clusters, at least 7 clusters, at least 9 clusters, at least 10 clusters, or at least 15 clusters. In some embodiments, the iRF model is used to determine the most important SVs for each cluster, and the most important SVs are matched to phenotype or treatment outcomes.

Gini importance is one such importance score method that captures how well a feature is able to split nodes in the random forest trees such that the child nodes contain more ‘pure’ samples than the parent node did. In some embodiments, from the ranked features (SVs) list produced by the iRF model, top N (where N can be any arbitrary number) SVs are selected. In some embodiments, the selected top SVs are genic (meaning they correspond to or occur in a specific gene), thereby providing a top N list of genes that are most important for modeling whether a case should belong to a specific cluster or not. This same process can be performed for each cluster, resulting in a unique list of top N genes for each cluster.

In some embodiments, the processor is further programmed for determining the frequency an NMI and comparing the frequency of NMIs at the corresponding genomic location in a population, and determining that the NMI indicates a structural variation if the frequency of the NMI is higher than that of the corresponding genomic region in the population.

In some embodiments, the biologically important structural variations are selected from structural variations that reside in a gene in which less than 5% of normal individuals have a known structural variation; and there is a run of at least four SNPs with NMI in a row.

In some embodiments, identifying conserved regions of the genome is performed by a custom correlation coefficient (CCC) analysis as described herein.

In some embodiments, the processor is further programmed for assigning a probability score for having a run of NMI greater than 4.

In some embodiments, the processor is further programmed for removing NMI attributable to high levels of masked repetitive elements.

In some embodiments, the processor is further programmed for identifying pinpoint locations of the structural variations and identifying pinpoint locations of conserved blocs of genetic information.

In some embodiments, the processor is further programmed for using the locations of the structural variations and the locations of the conserved blocs of genetic information to identify locations of rare structural variations in genes that have conserved blocs of genetic information.

### Computer-Readable Storage Device

An aspect of this disclosure is directed to a computer-readable storage device, comprising instructions to perform: assembling single nucleotide polymorphism (SNP) data from parents and their offspring; analyzing the SNP data for a plurality of non-Mendelian inheritance (NMI) patterns, wherein each NMI in the plurality of NMI patterns is a potential structural variation; scoring the NMIs to identify large structural variations, wherein the presence of at least three neighboring SNPs that demonstrate NMI in the offspring indicates a large structural variation; removing SNPs that demonstrate NMI in the offspring but that overlap with at least one known existing variation; identifying conserved regions of the genome to filter regions that should be conserved but include a structural variation; identifying biologically important structural variations; and classifying the identified biologically important structural variations using a machine learning algorithm.

In some embodiments, the machine learning algorithm is a neural network.

In some embodiments, the machine learning algorithm is an iterative Random Forest (iRF). Iterative Random Forest is an improvement over standard Random Forest for datasets with large feature space. It applies feature-selection and boosting to iteratively remove noise and boost true signal. It therefore improves the reliability of the top-ranked (most important) features in the model. In some embodiments, it means that the genes that are determined to be most predictive of each disease cluster are probably more reliable than the equivalent result provided by Random Forest. In some embodiment, the iRF comprises assigning individuals in a single predefined cluster the value of 1, and the rest the value of 0. In some embodiments, the single predefined cluster comprises individuals diagnosed with a particular disease (e.g., ASD, MS etc.) and the rest of the individuals are people not diagnosed with the disease. In some embodiments, the presence/absence for each gene or genomic region is set to 0/1, respectively, and all genes are used as features in the iRF model, which performs an iterative feature selection. In some embodiments, this process is repeated for each of the clusters, resulting in a final random forest for each cluster. In some embodiments, top 10, top 15, top 20, top 25, or top 30 most important genes or genetic regions for each cluster are extracted based on their Gini importance scores provided by the Ranger v0.12 R package.

In some embodiments, clusters of a disease (i.e., groups of cases that are more similar to each other based on which SVs they have) are defined through unsupervised learning algorithms. For a given cluster, all cases in that cluster are given a value of 1, while all ASD cases outside the cluster are given a value of 0. An iRF model is then trained using the SV presence/absence input matrix as features in order to explain the 0 or 1 cluster assignments of the cases. Once the iRF model is fit, the importance score of each input feature (SV) can be obtained so that the SVs can be ranked from most important to least important according to the model.

In some embodiments, there are at least 3 clusters, at least 4 clusters, at least 5 clusters, at least 6 clusters, at least 7 clusters, at least 9 clusters, at least 10 clusters, or at least 15 clusters. In some embodiments, the iRF model is used to determine the most important SVs for each cluster, and the most important SVs are matched to phenotype or treatment outcomes.

Gini importance is one such importance score method that captures how well a feature is able to split nodes in the random forest trees such that the child nodes contain more ‘pure’ samples than the parent node did. In some embodiments, from the ranked features (SVs) list produced by the iRF model, top N (where N can be any arbitrary number) SVs are selected. In some embodiments, the selected top SVs are genic (meaning they correspond to or occur in a specific gene), thereby providing a top N list of genes that are most important for modeling whether a case should belong to a specific cluster or not. This same process can be performed for each cluster, resulting in a unique list of top N genes for each cluster.

In some embodiments, the processor is further programmed for determining the frequency an NMI and comparing the frequency of NMIs at the corresponding genomic location in a population, and determining that the NMI indicates a structural variation if the frequency of the NMI is higher than that of the corresponding genomic region in the population.

In some embodiments, the biologically important structural variations are selected from structural variations that reside in a gene in which less than 5% of normal individuals have a known structural variation; and there is a run of at least four SNPs with NMI in a row.

In some embodiments, identifying conserved regions of the genome is performed by a custom correlation coefficient (CCC) analysis as described herein.

In some embodiments, the processor is further programmed for assigning a probability score for having a run of NMI greater than 4.

In some embodiments, the processor is further programmed for removing NMI attributable to high levels of masked repetitive elements.

In some embodiments, the processor is further programmed for identifying pinpoint locations of the structural variations and identifying pinpoint locations of conserved blocs of genetic information.

In some embodiments, the computer-readable storage device further comprises instructions for using the locations of the structural variations and the locations of the conserved blocs of genetic information to identify locations of rare structural variations in genes that have conserved blocs of genetic information.

### Methods Directed to Specific Genes

An aspect of this disclosure is directed to a method comprising: obtaining a biological sample from a subject, detecting in the biological sample whether at least one gene or genomic region selected from Table 1 and or Table 2 has a structural variation; and determining that the subject is at risk of Autism Spectrum Disorder if the at least one gene or genomic region has a structural variation.

TABLE 1
100 most frequent ASD-SVs and their locus details. OREG = Regulatory elements from ORegAnno,
cCRE—Candidate Cis-Regulatory Element. f(M) = frequency in MIAMI; f(A) = frequency in AGPC.

| rsID | Chrom | Pos | Locus/Gene | f(avg) | information |
|---|---|---|---|---|---|
| rs1867411 | chr12 | 59054013 | AC068305.2 | 0.73 | ncRNA |
| rs322461 | chr3 | 120808490 | Intergenic | 0.58 | Intergenic |
| rs12087237 | chr1 | 3646950 | WRAP73 | 0.55 | Neurotransmitter release |
| rs1316535 | chr6 | 149096780 | OREG1226770 | 0.55 | Intergenic |
| rs4923849 | chr15 | 34981419 | ZNF770 | 0.53 | Unknown |
| rs4396083 | chr1 | 188479092 | OREG1583503 | 0.52 | Intergenic |
| rs2085462 | chr19 | 35145952 | AC020907.2 | 0.52 | ncRNA |
| rs1554115 | chr3 | 106928636 | LINC00882 | 0.51 | ncRNA |
| rs9807181 | chr18 | 10590946 | Intergenic | 0.51 | Intergenic |
| rs497552 | chr7 | 105747278 | ATXN7L1 | 0.50 | A paralog of this gene, ATXN7 (Ataxin |
| | | | | | 7), is associated with Spinocerebellar |
| | | | | | Ataxia. |
| rs2595243 | chr3 | 159277160 | IQCJ-SCHIP1 | 0.49 | Axon guidance |
| rs11083725 | chr19 | 43796424 | LYPD5 | 0.48 | Unknown |
| rs7787574 | chr7 | 55817981 | SEPT14 | 0.48 | Dendritic spinogenesis |
| rs6905201 | chr6 | 31375450 | AL671883.3 | 0.48 | ncRNA |
| rs4699965 | chr5 | 60872061 | ERCC8 | 0.47 | DNA repair by non-homologous end |
| | | | | | joining (NHEJ) |
| rs7214288 | chr17 | 41085708 | KRTAP9 | 0.47 | Keratin fibers |
| rs8067444 | chr17 | 22589809 | Intergenic | 0.47 | Intergenic |
| rs4856657 | chr3 | 161848811 | Intergenic | 0.47 | Intergenic |
| rs11120900 | chr1 | 7318120 | CAMTA1 | 0.47 | Transcription |
| rs2316539 | chr7 | 154935298 | PAXIP1-AS2 | 0.47 | ncRNA |
| rs12714190 | chr2 | 86553038 | CHMP3 | 0.47 | Endosomal sorting |
| rs1910384 | chr4 | 165957402 | TLL1 | 0.47 | Protease influences dorsal-ventral |
| | | | | | patterning and skeletogenesis. |
| rs9914195 | chr17 | 82426276 | HEXD | 0.46 | Has hexosaminidase activity |
| rs7793367 | chr7 | 6578314 | ZDHHC4 | 0.46 | Palmitoyltransferase that adds palmitate |
| | | | | | ontoD(2) dopamine receptor DRD2. |
| rs9307811 | chr4 | 83002716 | LIN54 | 0.46 | Transcription |
| rs9479405 | chr6 | 150016929 | OREG11844 | 0.45 | Intergenic |
| rs2072707 | chr22 | 36923039 | CSF2RB | 0.45 | Interleukin-3 receptor |
| rs7797117 | chr7 | 77147083 | AC007000.3 | 0.45 | ncRNA |
| rs4819061 | chr21 | 45315832 | Intergenic | 0.45 | Intergenic |
| rs7155109 | chr14 | 39623141 | AL049828 | 0.45 | ncRNA |
| rs13188943 | chr5 | 45804033 | Intergenic | 0.45 | Intergenic |
| rs17344051 | chr2 | 212082631 | ERBB4 | 0.44 | Axon guidance |
| rs469942 | chr5 | 94911548 | MCTP1 | 0.44 | Neurotransmitter release |
| rs9847153 | chr3 | 147642163 | Intergenic | 0.44 | Intergenic |
| rs12549801 | chr8 | 141431542 | PTP4A3 | 0.44 | Protein tyrosine phosphatase |
| rs1012066 | chr1 | 178995469 | Intergenic | 0.44 | Intergenic |
| rs249223 | chr5 | 80547946 | Intergenic | 0.44 | Intergenic |
| rs6925697 | chr6 | 44465593 | Intergenic | 0.44 | Intergenic |
| rs440091 | chr4 | 107099752 | DKK2 | 0.43 | Inhibits Wnt regulated antero-posterior |
| | | | | | axial patterning. |
| rs7258495 | chr19 | 39792268 | LEUTX | 0.43 | Homeobox transcription factor involved |
| | | | | | in embryogenesis |
| rs1960049 | chr4 | 115208964 | Intergenic | 0.43 | Intergenic |
| rs13340529 | chr7 | 67001132 | TYW1 | 0.43 | Wybutosine biosynthesis pathway |
| rs2261567 | chr6 | 32786317 | Intergenic | 0.43 | Intergenic |
| rs3843752 | chr19 | 54631479 | LILRB1 | 0.43 | Receptor for class I MHC antigens. |
| rs11079480 | chr17 | 62515390 | TLK2 | 0.43 | Chromatin modification |
| rs3856834 | chr3 | 16540153 | LINC00690 | 0.43 | ncRNA |
| rs1254282 | chr14 | 60388905 | Intergenic | 0.43 | Intergenic |
| rs11780763 | chr8 | 128584040 | OREG1283103 | 0.43 | Intergenic |
| rs10185485 | chr2 | 126601491 | Intergenic | 0.43 | Intergenic |
| rs1829737 | chr7 | 63115622 | Intergenic | 0.43 | Intergenic |
| rs2038067 | chr6 | 35406689 | PPARD | 0.42 | Regulates the peroxisomal beta- |
| | | | | | oxidation pathway of fatty acids |
| rs2126389 | chr1 | 223874196 | OREG1262585 | 0.42 | Intergenic |
| rs3094710 | chr6 | 30385292 | GL000255v2_alt | 0.42 | Intergenic |
| rs4104504 | chr4 | 107290249 | AC104663.1 | 0.42 | ncRNA |
| rs8023343 | chr15 | 56927576 | TCF12 | 0.42 | Initiates neuronal differentiation |
| rs946786 | chr10 | 6954460 | AL392086 | 0.42 | ncRNA |
| rs1544631 | chr12 | 4206485 | EH38E1588492 | 0.42 | Intergenic |
| rs2163842 | chr19 | 45045244 | CLASRP | 0.42 | Splicing regulator |
| rs1333939 | chr9 | 80356912 | Intergenic | 0.42 | Intergenic |
| rs12656368 | chr5 | 180226428 | AC104115.1 | 0.42 | ncRNA |
| rs4547037 | chr10 | 85674716 | GRID1 | 0.42 | Glutamate signaling |
| rs10404960 | chr19 | 18948676 | HOMER3 | 0.41 | Glutamate signaling |
| rs1387910 | chr6 | 123840406 | NKAIN2 | 0.41 | Interacts with sodium/potassium- |
| | | | | | transporting ATPase |
| rs9315483 | chr13 | 37346055 | Intergenic | 0.41 | Intergenic |
| rs12547271 | chr8 | 5072374 | Intergenic | 0.41 | Intergenic |
| rs6054459 | chr20 | 6689734 | OREG1291155 | 0.41 | Intergenic |
| rs7181542 | chr15 | 99626095 | MEF2A | 0.41 | Promotes synaptic differentiation |
| rs11943040 | chr4 | 128293222 | LINC02615 | 0.41 | ncRNA |
| rs10802632 | chr1 | 237764921 | RYR2 | 0.41 | Dendritic spinogenesis |
| rs966227 | chr10 | 113087007 | TCF7L2 | 0.40 | Wnt signaling |
| rs10244600 | chr7 | 16200529 | ISPD | 0.40 | Cytidylyltransferase required for protein |
| | | | | | O-linked mannosylation |
| rs1985332 | chr13 | 22941414 | Intergenic | 0.40 | Intergenic |
| rs2196516 | chr11 | 91265428 | Intergenic | 0.40 | Intergenic |
| rs2826833 | chr21 | 21432354 | NCAM2 | 0.40 | Axon guidance |
| rs4128796 | chr8 | 138163465 | FAM135B | 0.40 | Unknown |
| rs3976523 | chr3 | 179381391 | MFN1 | 0.40 | Mitochondrial fusion |
| rs3860912 | chr9 | 83386145 | FRMD3 | 0.40 | Four-point-one, ezrin, radixin, moesin |
| | | | | | (FERM) domain |
| rs11079808 | chr17 | 48102870 | CBX1 | 0.40 | Chromatin modification |
| rs17083190 | chr6 | 121117232 | TBC1D32 | 0.40 | Sonic hedgehog signaling for |
| | | | | | development of neural tube |
| rs11695642 | chr2 | 43725556 | PLEKHH2 | 0.40 | F-actin stabilizing |
| rs11697386 | chr20 | 5322817 | AL121757 | 0.40 | ncRNA |
| rs2432052 | chr19 | 36227484 | ZNF565 | 0.40 | Transcription |
| rs10185160 | chr2 | 25921564 | AluSq | 0.40 | TE |
| rs10939683 | chr4 | 16669771 | LDB2 | 0.40 | Transcription |
| rs9394827 | chr6 | 12392528 | Intergenic | 0.40 | Intergenic |
| rs7584086 | chr2 | 168786712 | CERS6-AS1 | 0.39 | ncRNA |
| rs9929889 | chr16 | 51060127 | MIR548AI | 0.39 | ncRNA |
| rs1567477 | chr4 | 178418326 | Intergenic | 0.39 | Intergenic |
| rs549287 | chr6 | 10799263 | TMEM14B | 0.39 | Development of neocortex |
| rs974176 | chr2 | 214304188 | SPAG16 | 0.39 | Necessary for sperm flagellar function |
| rs814376 | chr4 | 116111374 | AC027613.1 | 0.39 | ncRNA |
| rs4383556 | chr3 | 185666685 | IGF2BP2 | 0.39 | RNA-binding factor that recruits target |
| | | | | | transcripts to cytoplasmic protein-RNA |
| | | | | | complex |
| rs34270714 | chr1 | 223101459 | AL929091 | 0.39 | ncRNA |
| rs4365863 | chr5 | 96101907 | AC104123.1 | 0.39 | ncRNA |
| rs6694490 | chr1 | 205837051 | PM20D1 | 0.39 | Regulates the endogenous N-fatty acyl |
| | | | | | amino acids |
| rs7983971 | chr13 | 52216565 | AL158066.1 | 0.39 | ncRNA |
| rs9385601 | chr6 | 132342322 | MOXD1 | 0.39 | A paralog of this gene, DBH, catalyzes |
| | | | | | the conversion of dopamine to |
| | | | | | norepinephrine |
| rs10858939 | chr12 | 89911575 | AC084200.1 | 0.39 | ncRNA |
| rs9481031 | chr6 | 110021977 | Intergenic | 0.39 | Intergenic |
| rs1381342 | chr18 | 42211268 | LINC00907 | 0.39 | ncRNA |

TABLE 2
Most important genes containing ASD-SV according
to iterative Random Forest classifiers.

| Group1 | Group2 | Group3 |
|---|---|---|
| PRKD1 | ZNF208 | CACNA2D1 |
| CTNNA2 | HBS1L | PACRG |
| PPM1E | MAGI2 | PIEZO2 |
| | | CNST |

In some embodiments, the method comprises determining that the subject is at risk of Autism Spectrum Disorder if at least 2, at least 3, at least 4, at least 5, at least 10, at least 20, at least 25, at least 30, at least 35, at least 40, at least 45, at least 50, at least 55, at least 60, at least 65, at least 70, at least 75, at least 80, at least 85, at least 90, at least 95 or all the genes or genomic regions in Table 1 and/or Table 2 comprise a structural variation.

In some embodiments, the at least one gene comprises the glutamate ionotropic receptor kainate type subunit 2 (GRIK2) gene (OMIM No: 138244, NCBI Gene ID: 2898).

An aspect of this disclosure is directed to a method comprising: obtaining a biological sample from a subject, detecting in the biological sample whether the GRIK2 gene has a structural variation; and determining that the subject is at risk of Autism Spectrum Disorder if the GRIK2 gene has a structural variation.

In some embodiments, the at least one gene comprises the aminocarboxymuconate semialdehyde decarboxylase (ACMSD) gene (OMIM No: 608889, NCBI Gene ID: 130013).

An aspect of this disclosure is directed to a method comprising: obtaining a biological sample from a subject, detecting in the biological sample whether the ACMSD gene has a structural variation; and determining that the subject is at risk of Autism Spectrum Disorder if the ACMSD gene has a structural variation.

Unless defined otherwise, all technical and scientific terms used herein have the same meaning as commonly understood by one skilled in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

The specific examples listed below are only illustrative and by no means limiting.

### EXAMPLES

### Example 1: Materials and Methods

### Samples and Quality Control

Array-based genotypes from ASD cases and their parents were obtained from the database of Genotypes and Phenotypes (dbGaP). For SV discovery, the inventors used a dataset from an ASD study from the University of Miami consisting of 1,177 individuals that represent 381 families genotyped at 1,048,847 nuclear SNP loci (dbGAP accession phs000436.v1.p1). The inventors labeled this dataset as MIAMI. For validation, the inventors used data from a second study, which was produced by the Autism Genomic Project Consortium (AGPC), and consists of 4,168 individuals representing 1,385 families genotyped at 1,072,657 nuclear loci (dbGAP accession phs000267.v5.p2). The inventors labeled this dataset as AGPC. Data were handled in accordance with the rules established by the National Institutes of Health. Potentially erroneous SNPs were removed by excluding all those with a quality score of less than 0.75, and the inventors performed a kinship analysis to ensure there was no overlap between individuals in MIAMI and AGPC.

### Non-Mendelian Inheritance (NMI) Detection and Re-Genotyping

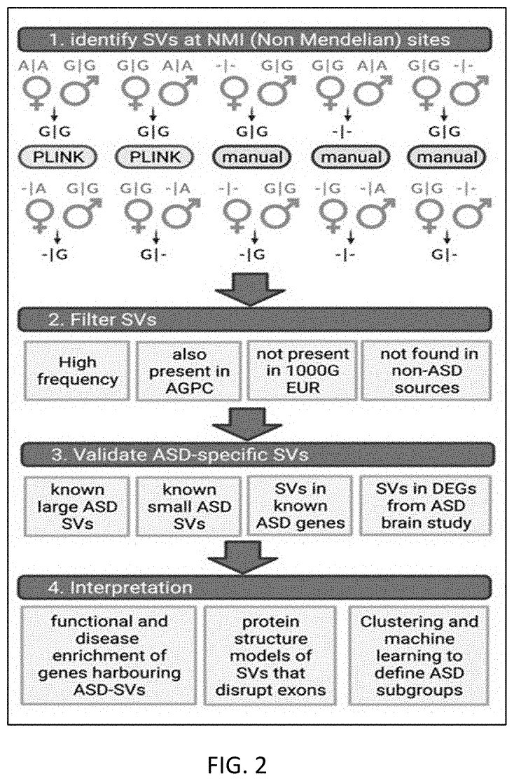

The inventors used the program PLINK v1.9 with the 890,539 autosomal SNPs that remained after QC filtering to identify loci that did not conform to Mendelian inheritance and therefore represent likely SV. The inventors did not include SNPs on the X chromosome because NMI cannot be determined on the X in males due to hemizygosity. In most cases of NMI that the inventors observed, the Mendelian expectation was that the child should be heterozygous at a site but instead displayed homozygosity (FIG. 2, FIG. S1). There are a considerable number of cases where an SV may exist and be causing erroneous genotype calls, but PLINK does not detect NMI at that site because all three members of the trio show the same homozygous genotype (e.g., all are A/A). However, if a number of other trios at that site do have clearly detectable NMI patterns, then the inventors can leverage their genotyping signal intensities to find SVs in individuals not called by PLINK (FIG. 1B, center). If an individual's genotype intensity co-located on a signal plot with those with NMI, then the inventors marked this as “suspected NMI” and can infer the presence of an SV in that individual. Once individuals were marked as NMI or suspected NMI at a site, the inventors re-genotyped them according to signal intensity plot positions (FIG. 1b, right).

The mendel function in PLINK outputs codes that can be directly translated into paternal or maternal errors. In addition, some NMI trio genotype combinations are ignored by PLINK, so these were scored manually and combined with the scored sites into a single matrix of genotypes for each of MIAMI and AGPC. For example, the inventors scored scenarios where genotypes were child=“A/A”, father=“A/A”, and mother=“−/−”, assigning it as a maternal SV. Paternal SV was assigned when the genotype is missing for the father but present in the mother. In this matrix the sites represent putative SVs of indeterminate length, though an upper bound of length can be derived by observing the basepair distance to the next normal mendelian site on the array. The NMI genotyping workflow can be seen in FIG. 2. The inventors used the smaller MIAMI data set (N=381 families) for SV discovery and the large AGPC data set (N=1136 families) for validation.

### Filtering NMI SVs

The instant goal was to reduce the initial set of NMI sites to a set of reliable ASD-specific SVs that are most likely to represent the core of the missing heritability of ASD. FIG. 2 provides an overview of the workflow described below.

First, the inventors applied filters to remove potential false positive SV genotypes. Rarer SVs are more likely to be due to error than common SVs, so the inventors removed all SVs with frequency less than 2% in the discovery population (MIAMI). The inventors chose 2% because this is the estimated frequency of ASD in humans. It is also an extremely conservative filter given that the technical error rate for the Illumina array used in this study was estimated to be less than 0.05%. A potential cause of a false positive genotype for an array SNP is the presence of other SNPs in the immediate genomic region of the probe for that SNP. Therefore, the inventors also removed any SV whose probe overlapped another SNP (according to dbSNP153) with a MAF>0.02 in the 1000G EUR population. Finally, SVs that are found in only the discovery dataset are more likely to be false positives, so the inventors intersected the NMI SVs discovered in the MIAMI population with those in the AGPC validation population and removed any which did not appear in both. The resulting set of higher confidence SVs was labeled as NMI-SV.

Next the inventors reduced the NMI-SV set to a subset of novel ASD-specific SVs by removing those whose genotyping probe intersected with previously identified SV intervals with MAF>0.02 in one or more non-ASD-specific sources. Sources included the 1000 Genome Project hg38, a long-read sequencing scan from the same population, 433,371 SVs identified from 14,891 diverse genomes, and a recent report of 107,590 SVs (most of them novel) from genome-scale resolved haplotypes. To be conservative, the inventors removed NMI-SVs in this manner even if they resided in a gene that had previously been identified as ASD-related (see NRXN3, FIG. 1). The NMI-SVs that appeared in both ASD study populations and passed through all filters were labeled as ASD-SVs. Finally, the inventors reasoned that the core biological pathways in ASD would be represented by the most frequent ASD-SVs, so the inventors defined a core set of ASD-SVs found in both study populations at greater than 15% frequency.

### Detecting Large SVs

To identify large SV (runs of NMI in each individual), the inventors calculated a running sum on position-sorted NMI with a window size of 5 SNPs and calculated the probability of obtaining 5 sequential NMI SNPs on arrays that were randomized, i.e., SNPs that are adjacent on a chromosome are spread randomly across each array. The binomial probability of obtaining 5 successes (k) in 5 trials (n) with a probability of success of 0.36% (p) is 6×10⁻¹³.

Gene Enrichment

The set of genes harboring NMI-SVs were subjected to enrichment tests to determine if they were functionally non-random. The inventors used a chi-square test to see if these genes were enriched for ASD-susceptibility protein-coding genes listed in both SFARI (sfari website in April 2021) and AutDB (autism database in April 2021) databases.

The set of genes harboring core ASD-SVs (those with freq>15% in both populations) were assessed for enrichment for Gene Ontology biological process (GO BP) terms with a false discovery rate (FDR)<0.05. Additionally, the inventors performed a permutation test by computing GO enrichment on 100 randomly sampled sets of 1,106 genes from a list of all genes that overlapped SVs identified from fully-resolved genome wide-haplotypes in the 1000 Genome population (N=5,810 protein coding genes). Functional analyses for specific genes were taken from GeneCard Human Gene Database. ToppGene (ToppGene website) was used for the disease associated enrichment test of the core ASD-SV genes.

### Gene Expression Analysis for GRIK2

The inventors downloaded RNA-seq FASTQ files for 13 ASD cases and 10 controls from bulk prefrontal cortex listed in project PRJNA434002 in the sequence read archive (SRA) at NCBI. Reads were trimmed with CLC Genomics Workbench (version 20.0.4) then mapped to the human transcriptome GRCh38_latest_rna.fa with the following modifications: (1) predicted mRNA sequences were removed (those with the prefix “XM”), (2) all GRIK2 transcripts were removed and replaced with a single transcript containing only exons 11, 12, and 13. This was done to reduce bias from reads mapping to UTRs and to focus on potential loss of exon 12 because this is the exon adjacent to the ASD-SV and predicted to be lost from aberrant splicing. Mapping parameters were set to 0.95 for both length fraction and similarity fraction to reduce mis-mapping of reads from closely related genes (e.g., GRIK1 and GRIK2). The CLC Genomics tool Differential Expression for RNA-Seq was used with TMM normalization to control for library sizes. This tool assumes a negative binomial distribution for read counts similar to EdgeR and DESeq. Correlation between PTPRD and GRIK2 expression was determined with a Pearson correlation test in the R package Hmisc. Significance was determined with an FDR correction<0.05.

### Association Testing for Verbal/Non-Verbal Forms of ASD

In order to perform a Genome Wide Association Study using ASD-SVs the inventors first collapsed all ASD-SV sites within a gene's boundaries (according to RefSeq) to a single presence/absence marker. If at least one of the ASD-SVs sites in a gene was present for an individual, then an ASD-SV was considered as present in that gene, even if the other sites were absent. Those sites that were not assigned to a gene by RefSeq were annotated with their rsID, and loci found at less than 5% frequency were removed, leaving 10,108 presence/absence markers for further analyses. The inventors performed a logistic regression in PLINK and used the first two components of a PCA generated from 42,761 neutral SNPs as covariates to account for substructure of the ASD population (Supp Methods). The verbal (control) and non-verbal (case) phenotypes were extracted from the meta data included with the dbGAP project.

### Classification of ASD Subtypes Based on Genic ASD-SVs

By collapsing core ASD-SVs within gene boundaries, the inventors obtained presence/absence markers in the larger AGPC population for 1106 genes with frequency 15%. Sub-structure within the presence/absence matrix was visualized in two dimensions using tSNE in R. The inventors then applied hierarchical clustering using hclust with Bray-curtis distance and ward.D2 method in R, and selected clearly defined clusters as putative subtypes of ASD. In order to determine which genes have presence/absence patterns that define these subtypes, the inventors used a custom R implementation of iterative Random Forest (iRF) machine learning to classify the cluster labels. To do so, the inventors set the labels for individuals in a single cluster to 1, and the rest to 0. The presence/absence for each gene was set to 0/1 and all genes were used as features in the iRF model, which performs an iterative feature selection. This process was repeated for each of the clusters, resulting in a final random forest for each cluster. The top 10 most important genes for each cluster were extracted based on their Gini importance scores provided by the Ranger v0.12 R package.

### Sample Processing

Potentially erroneous SNPs were removed by excluding all assays with a quality score of less than 0.75. One family was removed from the Miami data set and two from AGPC due to poor data quality and 248 families were removed from AGPC because they did not have a quality score listed with the genotypes or were not part of a trio (i.e., those missing one or both parents). In order to ensure the inventors were analyzing two independent sets of parent-child trios, the inventors performed a kinship analysis on all of the individuals from the 380 families from the University of Miami study and the 1,136 families from the AGPC study. The inventors randomly chose 50,000 SNPs that conformed to Hardy-Weinberg-Equilibrium (HWE) and Mendelian inheritance, and had a minor allele frequency (MAF) of greater than 0.05. The inventors also pruned SNPs that had an LD>0.20 using the default step and window size on PLINK 1.9. The inventors then removed any SNPs in which alleles were INDELs, A/T or G/C pairs, or were found on the pseudoautosomal regions of the sex chromosomes, leaving 48,478 SNPs for further analysis. The inventors used the KING function in PLINK2 to estimate kinship. Kinship estimates within families were as expected. The inventors identified a single female that was listed in two different trios within the AGPC study, which was consistent with the metadata as she was the mother in different trios (different fathers). No individuals were identified among trios that would indicate overlap of the Miami and AGPC data sets. In order to identify potential substructure of the ASD population, after excluding all loci that demonstrated NMI as potential SVs, the inventors randomly chose 50,000 SNPs from the remaining assays. After intersecting with the 1000 Genome population and excluding those with MAF<0.05, the inventors retained 42,761 for the PCA performed in PLINK.

### High-Confidence SVs

Using the Miami and AGPC datasets, the inventors performed an NMI test in PLINK on both sets of data, which flagged 101,032 SNPs having at least one family with NMI in one of the data sets. The inventors then manually scored these 101,032 sites for NMI in further families that PLINK did not flag and estimated the frequencies within each population. All SVs found at a frequency of less than 2% in the Miami set were removed, leaving 61,703 as our discovery panel. The inventors chose 2% because this is the estimated frequency of ASD in the human population but also an extremely conservative filter given that the technical error rate for the Illumina array used in this study was estimated to be less than 0.05%. The 2% NMI rate corresponds to 7 individuals from the 380 families. The binomial probability of having a SNP assay fail 7 times in 380 trials given the technical error rate of 0.05% is 1.4×10⁻⁹, where p=0.05, n=380, and k=7. It should be noted that the quality control of the Illumina bead arrays releases assays that display the technical error rate of 0.05% or less, i.e., it does not account for error rate due to the samples being analyzed. Therefore, by definition, the error rate of 2% is conservative given that it is 40 times higher than technical background error.

Of this set, 90% (55,767) were found in at least one individual in the AGPC population. Next, the inventors used a Pearson correlation test with the rcorr function in the package Hmisc in the R programming environment and calculated a significant correlation between NMI SNPs in the discovery and validation data sets of 0.75 (p<0.0001). To identify large SV (runs of NMI in each individual), the inventors calculated a running sum on position-sorted NMI with a window size of 5 and calculated the probability of obtaining 5 sequential NMI SNPs on arrays that were randomized, i.e., SNPs that are adjacent on a chromosome are spread randomly across each array. There were a total of 338,404,820 genotyping assays in the Miami data set (380 families×890,539 SNPs used). Of these, 1,227,413 displayed an NMI pattern, or 0.36% of total genotyping assays across the 380 arrays. The binomial probability of obtaining 5 successes (k) in 5 trials (n) with a probability of success of 0.36% (p) is 6×10⁻¹³.

### ASD-Associated CNV

The AutDB CNV database was filtered for all cases with an ASD diagnosis for which there were genomic locations identified for the hg38 version of the human genome and overlapped at least one SNP from the Illumina Array and a genomic feature (N=22,233 cases). The inventors then intersected a BED file of these CNV with the ASD-SV to identify any that overlapped with the array. Because the inventors can already identify large CNV using runs of NMI SNPs, here the inventors wanted to focus on short CNV and therefore only included those that overlapped either one or two SNPs. CNV that overlapped a SNP with a minor allele frequency (MAF) of less than 0.001 were removed because they could not be discoverable with NMI. This left 2,270 CNV as a truth set. Of these, the inventors identified 1,902 with NMI (84%). Although the NMI proved to be a robust method to detect known CNV, the inventors wished to determine if lower allele frequencies of the SNPs that overlapped CNV could explain the inability to detect the remaining 16%. The inventors compared the MAF of the 1037 SNPs that overlapped the CNV that were successfully detected with NMI to the 207 SNPs that overlapped CNV yet were unable to detect them by NMI. Those SNPs that failed to detect CNV demonstrated a significantly lower MAF compared to those that succeeded (p<2.2×10⁻¹⁶, one-sided Wilcoxon rank sum test).

Differential Observed SV with GRIK2 ASD-SV at rs2051449

In order to determine if any ASD-SV were co-segregating with the one identified at rs2051449 in GRIK2, the inventors first plotted the genotypes using the original Illumina array intensity values as was done for the individuals at the NRXN3 SV_{NMI}. In this case, the pattern suggested that there were copy number gains linked to the A allele and the inventors therefore selected from the 1137 AGPC individuals the subset of those whose intensity value at the A allele was greater than those found in any of the heterozygotes. This is a conservative estimate of those with a gain because heterozygotes harbor only a single A allele and therefore intensities will be lower than homozygotes. The inventors calculated the expected number of each ASD-SV based on the overall frequency in the AGPC population (381 with and 756 without the ASD-SV at rs2051449) and tested for significance with a Chi-squared test. Because this test is unreliable at low numbers, the inventors only included ASD-SV that were found in at least 20 individuals. Of these 26,524 ASD-SV, 15 were found to be differentially observed (FDR<0.05). FDR was calculated using the p.adjust function in R with the Benjamini & Hochberg method. All significantly different ASD-SV were found at lower than expected numbers and two were identified in the same gene, PTPRD.

### Association Tests for Verbal/Non-Verbal Forms of ASD

In order to perform a Genome Wide Association Study using ASD-SV the inventors first collapsed all sites within a gene's boundaries (according to RefSeq) to a single locus. If at least one of the ASD-SV sites in a gene was present for an individual, then an ASD-SV was considered as present in that gene, even if the other sites were absent. Those sites that were not assigned to a gene by RefSeq were annotated with their rsID, and loci found at less than 5% frequency were removed, leaving 10,108 loci for further analyses. The inventors performed a logistic association in PLINK and used the first two components of the PCA generated from 42,761 neutral SNPs (see 1.1 Sample processing) as covariates to account for substructure of the ASD population. The verbal (control) and non-verbal (case) phenotypes were extracted from the meta data included with the dbGAP project.

### Classification of ASD Subtypes Based on Genic SVs

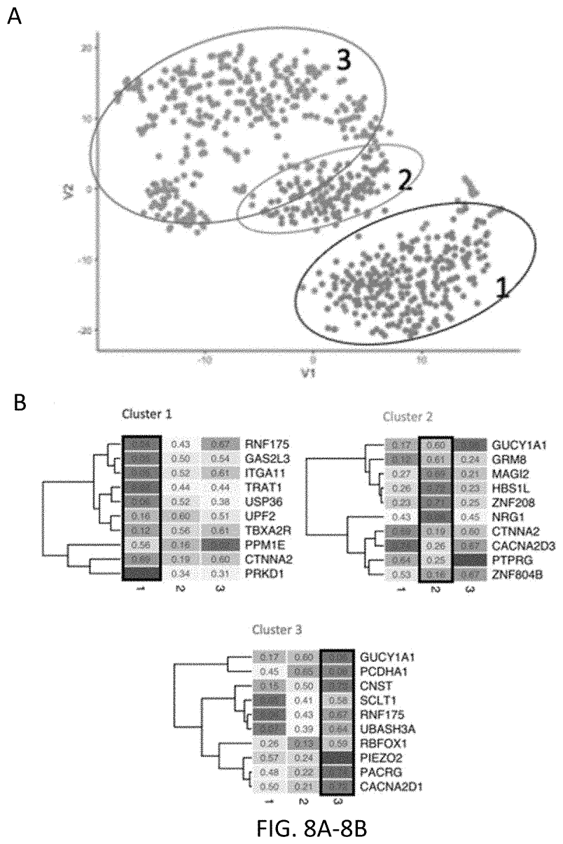

By collapsing ASD-SV sites within gene boundaries, the inventors obtained presence/absence markers in the AGPC population for 1106 genes with frequency>15%. Sub-structure within the presence/absence matrix was visualised in two dimensions using tSNE in R (FIG. 8A). The inventors then applied hierarchical clustering using hclust with Bray-curtis distance and ward.D2 method in R, and selected 3 clearly defined clusters as putative subtypes of ASD. In order to determine which genes have presence/absence patterns that define these subtypes, the inventors used a custom R implementation of iterative Random Forest (iRF) machine learning to classify the cluster labels. To do so, the inventors set the labels for individuals in a single cluster to 1, and the rest to 0. The presence/absence for each gene was set to 0/1 and all genes were used as features in the iRF model, which performs an iterative feature selection. This process was repeated for each of the three clusters, resulting in three final random forests. The top 10 most important genes for each cluster were extracted based on their Gini importance rankings.

### Example 2: Analysis

### SV Detection and Filtering

The inventors performed NMI tests in PLINK on both the MIAMI and AGPC datasets, which flagged 101,032 putative SV sites (i.e., having at least one family with NMI in one or both data sets). The inventors then manually scored these 101,032 sites for NMI in further families that PLINK did not flag and estimated the frequencies within each population (FIG. 2). Out of a total of 338.4 m genotyped sites in the MIAMI data set (i.e., 380 children x 890,539 SNPs used), 1.23 m displayed an NMI pattern, or 0.36% of total genotyping assays across the 380 arrays.

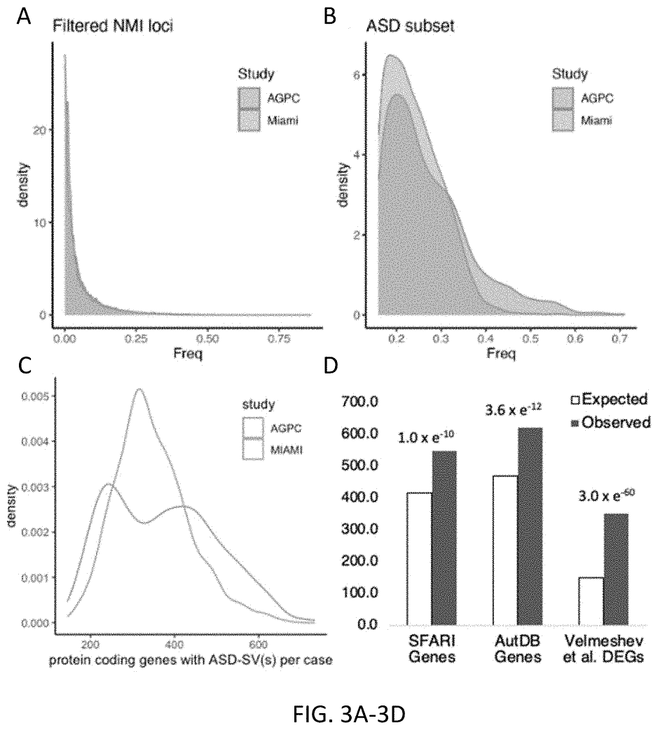

After removing rare SVs with frequency less than 2% in the MIAMI population, the inventors were left with 61,703 as the instant discovery panel. Of these, 55,767 (90%) were also detected as SVs in at least one family in the AGPC population (no individuals were present in both data sets, Supp Methods) (FIG. 3A). This set was labeled as NMI-SV. The frequencies of the discovery SVs in MIAMI were strongly correlated with those in AGPC (Pearson's r=0.75; p<0.0001), supporting the accuracy of this approach. To obtain the ASD-specific set of SVs the inventors next removed NMI-SV that were previously reported and known SVs from several sources including the 1000G (FIG. 2, see Methods). This left a total of 48,009 SVs in the ASD-SV set (5.5% of all sites in the array that passed QC) with frequency greater than 2% in the MIAMI population. The core of the ASD-SV set was defined by 1,175 SVs with greater than 15% frequency in both the MIAMI and AGPC populations, located in 1,106 protein-coding genes (FIG. 3B). On average, each individual in AGPC had 371 genes harboring high frequency ASD-SVs, while individuals in MIAMI averaged 347 (FIG. 3C).

### The NMI Method Strongly Recalls Known ASD-Related SVs

The SVs most confidently identified using the NMI method are those that represent large deletions that span multiple contiguous (on a chromosome) SNPs. The SNP loci are randomized on the array and therefore the probability of seeing NMI at each of these genomically contiguous SNPs by chance is extremely low. For example, the inventors identified NMI at 43 contiguous, physically linked SNPs in three individuals in the MIAMI data set. Based on the overall NMI rate across the array, the probability of finding this number of physically adjacent NMI loci due to technical error is exceedingly small (1.2×10⁻¹⁰⁵). Indeed, this particular stretch of 43 NMI SNPs most likely identifies a large SV that is known to cause subtypes of ASD including Angelman Syndrome (Pathania et al., 2014). By using these high-confidence consecutive NMI-SVs the inventors were able to identify 15 of the 17 ASD-susceptibility loci that are known to be large chromosomal disruptions.

To further test the instant approach, the inventors examined the SNPs that overlapped known ASD-associated copy number variation (CNV) SVs. The Autism DataBase (AutDB) lists CNV identified from the 28,735 ASD cases. Of the 2,270 small CNVs from AutDB that were potentially detectable with the SNPs on the Illumina array, the instant NMI approach captured 1,902 (84%) of them. This is a challenging test, since small CNVs overlap only one or two SNPs. Therefore, the result is highly supportive of the efficacy of NMI as a proxy for CNV detection.

### ASD Susceptibility Genes are More Likely to Harbour NMI-SVs

Of the 16,917 protein coding genes marked by the sites on the Illumina array, 49% (8,222) had at least one NMI-SV associated with them. The SFARI database lists 1,003 ASD-associated genes (see Data Description and Methods), of which 866 are marked by the Illumina array used in the MIAMI and AGPC studies. Assuming a random distribution of NMI-SVs across the genome, the instant expectation was that 421 of these genes would harbor an NMI-SV. However, the inventors found NMI-SVs in a significantly greater number (600, or 69%); (chi-square test p<2.5×10⁻¹⁸; FIG. 3D). Likewise, AutDB lists 1,241 ASD-associated genes, of which 1,072 are marked by the array used here. The inventors would expect to find 521 genes harboring NMI-SVs but, instead, the inventors find a significantly greater number (n=748, p<2.7×10⁻²³; chi-square test, FIG. 3D). The inventors see a similar enrichment when exploring 513 differentially expressed genes (DEGs) found in post-mortem brain tissue from ASD cases and controls. In this case, more than 70% of the DEGs (364 genes) harbor an ASD-SV, which is significantly greater than expected by chance (chi-square test, p<3.0×10⁻⁶⁰; FIG. 3D).

### Significant Functional Enrichment of Genes Harboring High Frequency ASD-SVs

To determine if the ASD-SVs were truly linked to the disorder, the inventors tested them for significant enrichment of biological process Gene Ontology (GO) terms. The inventors reasoned that the core biological pathways in ASD would be represented by the most frequent ASD-SVs, even in two unrelated ASD cohorts assembled for different purposes, therefore denoting the broad spectrum. To these ends, the inventors performed a GO enrichment analysis of characterized coding genes that harbor the core ASD-SVs in at least 15% of the cases (N=1,106). This resulted in four major significantly enriched biological processes (BP) (FDR<0.05, fold-enrichment>2), namely: dendritic spinogenesis, glutamate signaling, synaptic organization, and neuronal migration.

For further stringency the inventors performed GO analyses for each of 100 randomly sampled sets of 1,106 genes. Only 3/100 showed any enriched GO terms (FDR<0.01). Those 3 each returned only a single (BP) term, only one of which was related to neurobiology. In contrast, at the FDR<0.01 level, the core ASD-SV gene set returned the categories synapse organization, synaptic vesicle exocytosis, regulation of neuronal migration, and positive regulation of dendritic spine morphogenesis. The latter was nearly eight-fold enriched (FDR<0.007).

A disease ontology enrichment test using ToppGene returned highly significant diseases that included Autism and neurodevelopmental disorders (Bonferroni corrected p<2×10⁻¹³). Furthermore, the inventors intersected the instant core ASD-SVs with recently identified open chromatin regions of the developing human telencephalon (Markenscoff-Papadimitriou et al, 2020). This revealed that 118 core ASD-SVs also resided in open chromatin. A GO analysis of the 121 genes harboring those accessible SVs returned highly similar biological processes as the earlier analysis with 1,106 genes (FDR<0.05, fold-enrichment>2) and significant association with Autism Spectrum Disorder in TopGene (p<1.2×10⁻⁸, Bonferroni correction).

Finally, in order to identify the potential importance of SVs in intergenic and non-coding space, the inventors intersected the core ASD-SVs with transcription factor binding sites from the ENCODE database (ENCODE Project Consortium, 2012). ToppGene identified highly significant enrichment for the chromatin modifying and ASD-associated EMSY complex as well as lysine demethylases. EMSY was one of just two significantly differentially-expressed genes found in a transcriptome-wide association study of post-mortem brain tissue from individuals with ASD (Gupta et al, 2014).

Major Processes Disrupted by ASD-SVs Indicate they Represent Missing Heritability

Recent in-depth SV detection reports indicate there are roughly 28,000 SVs per individual in the human population. The inventors found that each ASD case had, on average, several hundred genes containing one or more high frequency ASD-specific SV (Miami=347, AGPC=371; FIG. 3C). Given the stringent filtering of the initial NMI-SVs, their validation in a second independent ASD dataset, and their high recall of known ASD-related SVs, these SVs are likely a key component of the spectrum of ASD. A GO enrichment analysis of coding genes that harbor the core ASD-SVs revealed significant enrichment of biological process terms involved in dendritic spinogenesis, glutamate signaling, synaptic organization, and neuronal migration. All have been repeatedly linked to ASD, supporting the hypothesis that these NMI-derived SVs represent a major component of the missing heritability of the disorder and is consistent with the heterogeneity of ASD because they indicate disruption of multiple components of a few different biological processes. In addition, because the instant method identifies narrow regions of the genome that are affected, the resulting gene set is of high-confidence and uncovers previously unknown links between these processes as well as an expanded set of genes that underlie the disorder.

It is clear from these analyses that the set of core ASD-SVs, obtained via the instant NMI workflow in a cohort of ASD trios, contains a strong neurobiological signal, and not by random chance. While previous ASD reports have identified many of the biological processes the inventors detected, only a handful of genes were attributed to these processes, and their seemingly diverse functions were attributed to pleiotropy. In contrast, here the inventors find subgroups of genes that define fine-grained biological networks within these processes and, more importantly, functional linkages amongst them that indicate that these seemingly functionally diverse genes actually converge on the central process of dendritic spine development in the cerebellum. The instant method also increases the number of genes associated with these biological pathways by nearly four-fold, further supporting the hypothesis that these loci represent the missing heritability of ASD. Table 1 presents the highest frequency ASD-SVs, and their relevant biological processes.

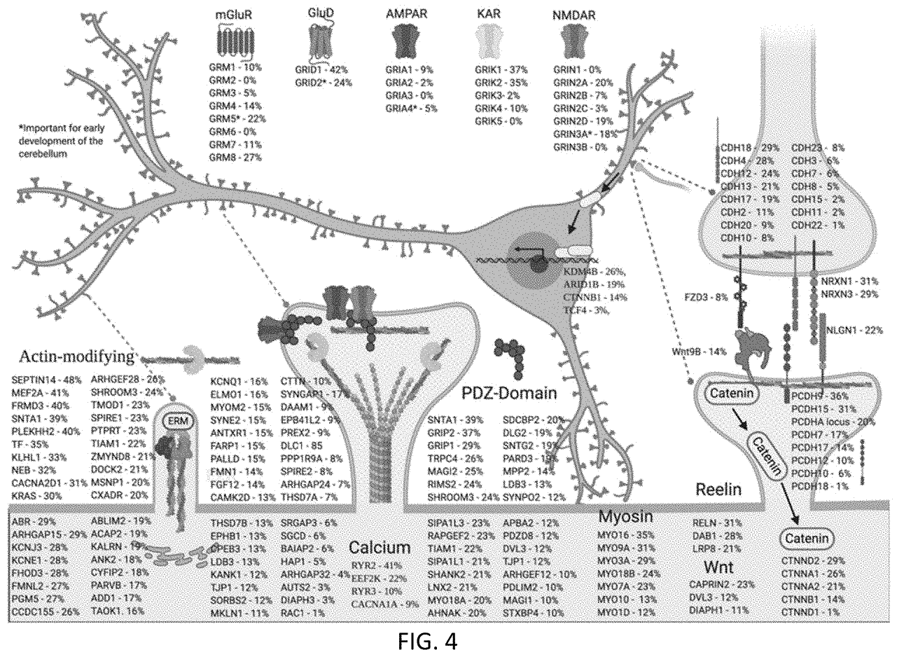

Dendritic spines are short protrusions that extend from the main shaft of a dendrite that play a central role in early brain development, neural plasticity, and long-term memory. These highly dynamic structures can rapidly change their shape and size and migrate in order to establish and dissolve synaptic connections with other neurons. Their dysfunction has been thoroughly described in ASD. The largest number of genes that are linked to these important structures are those that participate in their physical manifestation from the trunk of the neuron by altering the actin and myosin cytoskeleton (FIG. 4). The assemblage of genes the inventors identify using the instant method is a convenient demonstration of the molecular basis of the heterogeneity of a complex phenotype, i.e., how disruption of different genes can result in the alteration of the same biological function.

Of the 19 genes that are annotated with the GO BP term “positive regulation of dendritic spine morphogenesis” (GO:0061003), 8 of them contained high frequency ASD-SVs. For example, nearly one-fifth of ASD individuals carry an ASD-SV in the Kalirin gene (KALRN, rs2120789), which is a RhoGEF that has been associated with schizophrenia. Involvement of this gene in spinogenesis was confirmed by reports demonstrating its disruption in mice produces altered dendritic density. This enriched group also includes the RELN gene, which has been associated with ASD in more than 50 studies (SFARI), and also its associated receptor LRP8. Both genes harbor high frequency ASD-SVs and both are necessary for proper dendritic spine development. In addition to the group of eight genes returned by the GO analysis, the inventors obtained from literature a larger group of genes linked to dendritic spine morphogenesis (N=97) and supported by in vitro and in vivo work, many of which contain high frequency ASD-SVs. For example, the brain-specific Kelch-like protein 1 (KLHL1) has been shown to causes dendritic deficits in mice when mutated and copy number increases of the Necdin (NDN) gene, which lies at the terminal portion of the 15q11-q13 region the inventors identified with consecutive SV-NMI causes increased spine density and hyperactivity. Many others indirectly participate in the manipulation of the actin cytoskeleton by regulating Rho GTPases such as the genes encoding GTPase-activating proteins, ARHGAP24, ARHGAP15, and ARHGAP32, the last of which likely causes the ASD-like Jacobsen Syndrome.

Significant enrichment for the GO term “synaptic transmission, glutamatergic” (GO:0035249) highlights the involvement of glutamate signaling in ASD (FIG. 4). Glutamate receptors mediate excitatory synapse transmission in the brain and are grouped into five families (AMPAR, NMDAR, Kainate, Delta, and mGluR), all of which have been implicated in ASD and in the ASD-like Kleefstra Syndrome. Of the 26 genes that encode subunits of these receptors, the inventors find that 20 harbor an ASD-SV, many at high frequency (FIG. 4).

Importantly, a metabotropic glutamate receptor, GRM5 (mGluR5), initiates a cascade of events that are central to dendritic spine formation, strongly connecting the biological functions amongst the instant ASD-SVs. The inventors find that 22% of ASD cases harbor an ASD-SV in GRM5 (marked by rs1846476), which intersects and is therefore predicted to disrupt a FOXA1 binding site, suggesting that GRM5 is dysregulated in ASD individuals that carry this SV. Indeed, this was found to be the case in the recent single-cell RNA-Seq study.

The inventors find that several high frequency ASD-SVs reside in glutamate receptor subunits that are necessary for the early development of the cerebellum and are directly involved in development of the network of Purkinje cells and Climbing Fibers that are critical for the cerebellar function: GRM5 (22%), GRID2 (35%), GRIA4 (5%), and GRIN3A (18%) (Glutamate Signaling in Supplementary Text and FIG. 4). Further support is provided by an ASD-SV in GRIN2A that overlaps an open chromatin region necessary for fetal telencephalon development (rs6497523). Indeed, nearly all post-mortem examinations of ASD brains have found significant differences in the cerebellum compared to controls, including the loss of Purkinje cells, overall cerebellar enlargement early in development, and reduction in size by adulthood. Together with the dendritic spine morphogenesis genes, the disruption to glutamate signaling genes supports the hypothesis that ASD is likely a disorder centered around aberrant development of the cerebellum.

Finally, the enrichment for genes involved in neuronal migration buttresses the instant claim that these ASD-SV represent a substantial component of missing heritability and the genes the inventors identify interact with each other again supporting the claim that the heterogeneity of ASD results from disruption of different genes that participate in the same biological process. Live brain scans as well as post-mortem studies of ASD cases have identified an altered neuronal connectome. The development of complex neural circuits requires the migration of axons over long distances to make the appropriate connections to their target cells. This process requires an axon guidance “cone” at the tip, which senses attractant or repulsive cues secreted by astrocytes and other cells that lie along the path. The axons turn based on the combination of the molecule secreted and the receptor(s) being expressed at the tip of the cone. Upon passing a secreting sentinel cell, the receptors at the tip are degraded and replaced with new receptors that will sense the next decision point in the pathway. Often the axon will make contacts with the cell it passes via contactin and contactin-associated proteins (CNTNs and CNTNAPs).

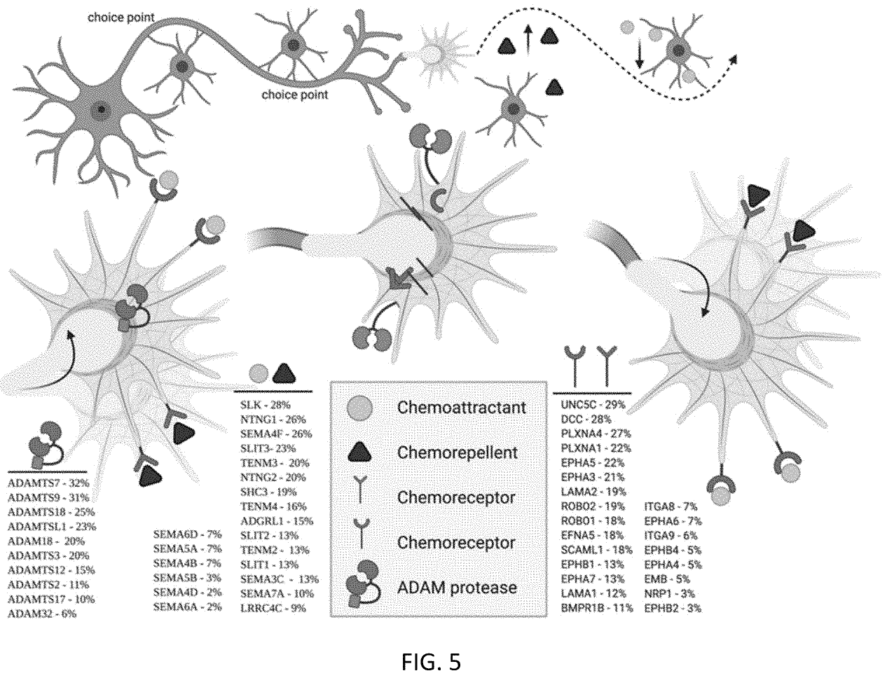

The majority of the axon-guidance related genes harboring ASD-SVs are either the receptors expressed at the cone of the migrating axon, or their partner ligand that is secreted by the cells at the choice point (See Axon guidance, FIG. 5). For example, the inventors identified frequent ASD-SVs in the Unc-5 Netrin Receptor C (UNCSD, rs4699836, 29% of cases), its cofactor DCC Netrin 1 Receptor (DCC, rs9304422, 28% of cases), and the ligand Netrin G1 (NTNG1, rs4915019 in 26% of cases), which has been associated with ASD and ASD-like RETT Syndrome. Similarly, two Roundabout Guidance Receptors (ROBO1 and ROBO2, rs4856257 and rs687813, 18% and 19% of cases respectively) and their ligands, Slit Guidance Ligands (SLIT3, SLIT2, SLIT1; rs7664347, rs888783, rs2636809 in 13%, 23%, and 13% of cases, respectively) carry ASD-SVs. Expression of ROBO1 and ROBO2 are significantly downregulated in ASD and SVs have been reported in ROBO2 in ASD cases. Variants in both ROBO3 and SLIT2 fully co-segregate with sound-color synesthesia (stimulation of one sensory input provokes perception in another), which is often comorbid with ASD. The distribution of ASD-SVs amongst several members of the same biological pathway and their previous association with the disorder are clearly non-random and provide even further support for the instant hypothesis that the NMI approach is identifying SVs that have previously gone undetected and explain missing heritability of ASD.

### Example 3

### Systems Biology Analysis and Functional Validation of a ASD-SV in the Kainate-Type Ionotropic Glutamate Receptor (GRIK2)

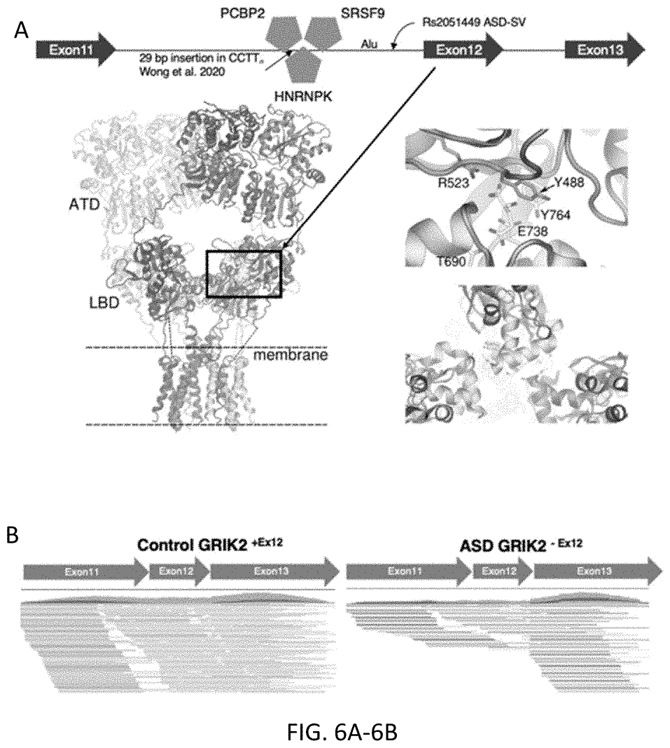

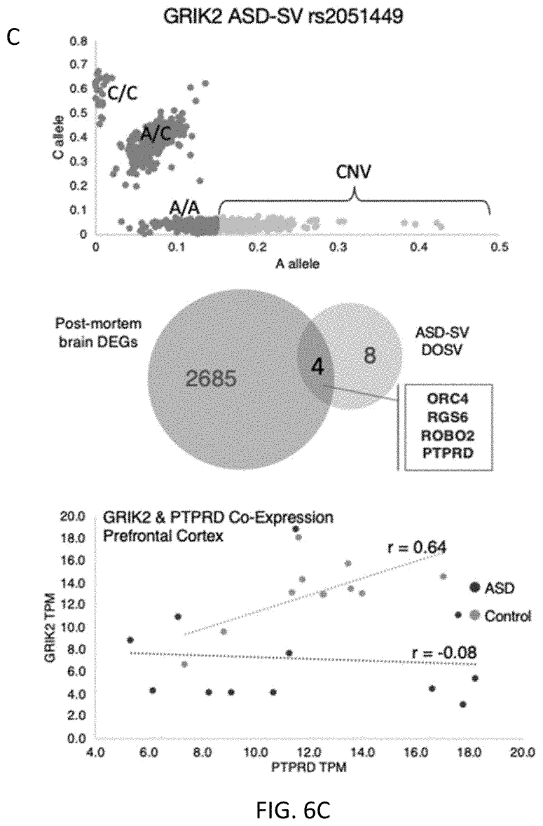

One of the most frequent ASD-SVs resides in the gene GRIK2, which encodes the GluK2 subunit of the kainate receptor (KAR, 35% of cases; FIG. 4) previously associated with ASD and, in line with convergence of ASD-SV to a few biological processes, is central to dendritic spine formation. The SNP (rs2051449) that marks this ASD-SV offers an opportunity to delve deeper into the genetic disruption linked to ASD because the NMI approach provides kilobase-resolution as to the locale of the SV. In this case, the ASD-SV overlaps a DNAse I hypersensitive site with a known CNV adjacent to exon 12 that binds an RNA-splicing complex (FIG. 6A). An SV at this site is therefore predicted to disrupt proper splicing of exon 12. Exon 12 codes for a portion of the glutamate binding pocket and therefore the loss of this exon would significantly disrupt glutamate signaling, especially as it is predicted to still be capable of assembling with other subunits via the preserved amino-terminal domains, which would result in a loss of function via a dominant negative mutation (FIG. 6A and Glutamate Signaling).

The predicted disruption of GRIK2 in ASD is supported by significant differential expression of GRIK2 in post-mortem brain tissue from ASD individuals compared to controls. However, that analysis was performed at the gene level. The inventors re-analyzed these data at the exon level, which revealed a roughly 50% reduction in transcripts within exon 12 in 10/13 ASD samples but in only one of the controls (FIG. 6B), thus providing stronger evidence of disruption of glutamate signalling in ASD due to an SV adjacent to exon 12.

To further interrogate the role of GRIK2 in ASD and find potential links to other ASD-SVs, the inventors first performed a differential gene expression analysis of the nine controls that retained GRIK2 exon 12 versus the ten ASD samples that showed reduced transcripts within GRIK2 exon 12. This identified 2,685 significantly differentially expressed genes (FDR<0.05; FIG. 6C). Similarly, the inventors split the AGPC data set into two sub-groups: those with and those without the SV at SNP rs2051449, based on a plot of the intensity values (FIG. 6C). The inventors identified 15 ASD-SVs that had significantly differentially observed frequencies (DOSV) between the two groups. Two of those ASD-SVs were in the PTPRD gene, whose mRNA was also found to be differentially expressed in the post-mortem prefrontal cortex in ASD individuals. Furthermore, both PTPRD and GRIK2 were previously identified in a GWAS as strongly associated with obsessive-compulsive disorder, which is highly comorbid with ASD. A plot of the expression of GRIK2 and PTPRD reveals that they are co-regulated in controls but not in ASD individuals (FIG. 6C).

As is the case with GRIK2, PTPRD regulates dendritic spine formation, further supporting the role of disruption of this process by SVs as core to ASD. Notably, the most frequent ASD-SV in PTPRD (rs7026388) lies within an exon, suggesting it disrupts the protein. It is highly noteworthy that most ASD individuals carry an ASD-SV either in PTPRD or in GRIK2, again consistent with the proposed molecular heterogeneity of the disorder, i.e., disruption of only one of those genes can result in ASD as they affect the same biological process.

ASD-SVs Provide an Important Marker Set for Association with Phenotype

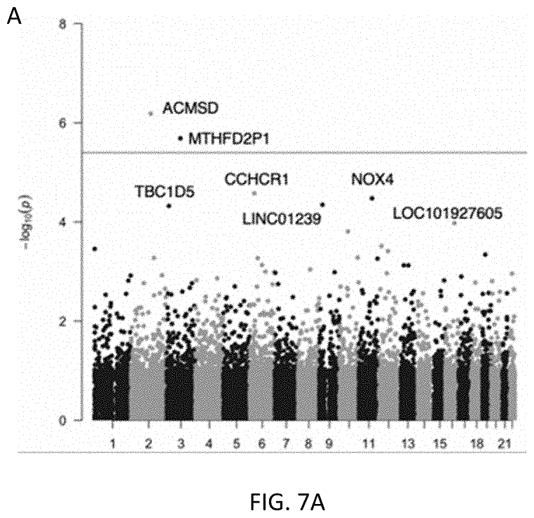

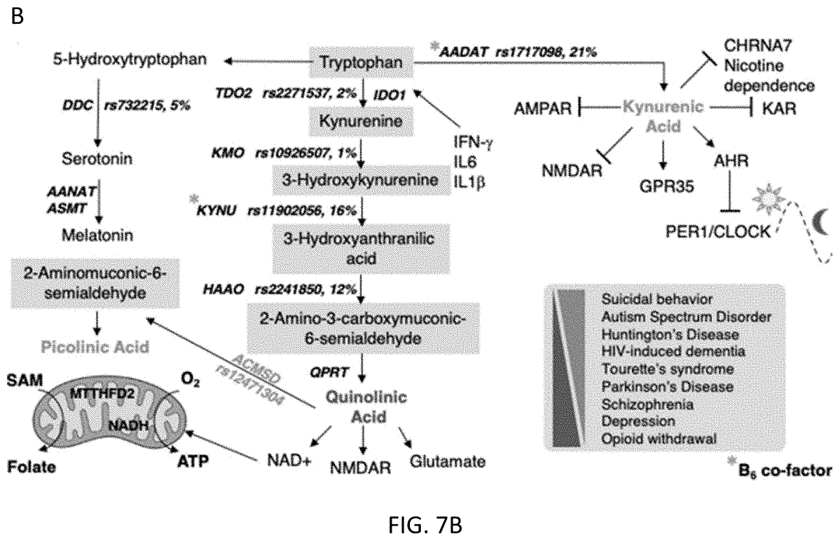

The inventors performed logistic association using a set of presence/absence markers encoded for ASD-SVs located within genes and verbal/non-verbal phenotype data. The test identified two significant loci, ACMSD and MTHFD2P1, after a conservative Bonferroni correction (p<5×10⁻⁶, FIG. 7a). ACMSD is an important enzyme in the tryptophan/kynurenine pathway, and is responsible for producing the neuroprotective picolinic acid from quinolinic acid substrate (FIG. 7b). Both the product and substrate have been linked to schizophrenia, Tourette's syndrome, epilepsy, depression, suicide, and importantly, ASD. Here, the significant ASD-SV occurs at a SNP (rs12471304) 1 kb from a FOS transcription factor binding site that has been reported to regulate the ACMSD gene in the Open Regulatory Annotation database (OREG1613578).

In addition to picolinic acid and quinolinic acid, tryptophan can also undergo catabolism to kynurenic acid through action of the enzyme aminoadipate aminotransferase (AADAT), which inhibits NMDA, Kainate, and AMPA receptors. A report of altered plasma levels of kynurenic acid and tryptophan in ASD cases compared to controls and correlation with disorder severity further supports the instant findings here. As is the case with picolinic acid, kynurenic acid appears to be neuroprotective (FIG. 7b). Notably, an ASD-SV at rs1717098 in AADAT is found in more than 20% of individuals in both the MIAMI and AGPC studies. The SV overlaps a regulatory site for AADAT, and a CNV in ASD cases has been reported in this gene. As with the biological pathways identified by the instant GO tests, the instant association test between verbal and non-verbal cases with only genomic regions harboring ASD-SVs pinpoint a specific pathway with multiple affected genes that has already been strongly associated with the disorder in previous studies.

### Clustering of ASD-SVs Reveals the Genetic Heterogeneity of Autism

By using an explainable artificial intelligence (X-AI) approach, the inventors demonstrate that the inventors can use the ASD-SVs to dissect the heterogeneity that has plagued past studies, providing further support that these genomic variants represent a large component of the missing heritability of ASD. Using hierarchical clustering the inventors were able to delineate several distinct sub-clusters of the AGCP ASD cases (FIG. 8a). Then, by using an iterative Random Forest classifier, the inventors identified the genes whose SV variation across the ASD cases most defined each cluster (FIG. 8b). This provides invaluable information for follow-up studies. For example, an ASD-SV in the CTNNA2 gene defines cluster number 1 and is associated with the startle response, whereas the CACNA2D1 gene, which defines cluster 3, is associated with Long QT cardiac arrhythmias. These NMI variants could be tested for association with distinct ASD phenotypes.

### Example 4

### Neurexin-3 NMI

The SNP rs221465 in the NRXN3 gene displays NMI in 35% of ASD individuals. This site is proximal to a ncRNA near an intron/exon border, a histone methylation site, and an enhancer that is expressed during neural tube development, making it an attractive candidate for ASD association. However, the most recent version of the human genome reported an 8.6 kb deletion at this location with an allele frequency of 0.28. After the Inventors re-scored the genotypes for this deletion in the GWAS population using the combination of raw intensity values and parental inheritance, the Inventors found normal Mendelian inheritance, conformation to Hardy-Weinberg Expectations, and no statistical difference from the 1000 Genome EUR population. This suggests that this SV is a false positive in the context of ASD, but also confirms that NMI is an accurate means to identify SVs based on information of normally segregating variants in the 1000 Genome population.

### Glutamate Signaling

The instant Gene Ontology analysis of the SV in coding regions identified several categories associated with glutamate signaling. Disrupted glutamate signaling has been thoroughly described in ASD and in the ASD-like Kleefstra Syndrome. Glutamate receptors mediate excitatory synapse transmission in the brain and were originally classified according to the glutamate analogs they bound. There are five families of receptors, all of which have been implicated in ASD. Four of the five function as transmembrane ion channels; these are known as ionotropic glutamate receptors or iGluRs. The fifth type are the metabotropic G-protein coupled glutamate receptors (mGluRs) and unlike the iGluRs, they respond through classic signal transduction pathways. All of these receptors are an important component of cerebellum function and development.

Even though the cerebellum comprises only 1/10th of the total brain volume, it is the most dense region and contains more neurons than the rest of the brain combined. Although this brain structure is most commonly associated with motor skills and physical movement, it also functions in the accurate coordination of motor skills as well as language processing and expression of emotion. Damage to different regions of the cerebellum results in impaired communication similar to ASD and cerebellar injury at birth increases the diagnosis of ASD by 36-fold. The cerebellum rapidly grows during the third trimester of pregnancy and differentiates early in development, but it is not mature until the first postnatal years. A highly organized network resides in the cerebellum that is composed of Climbing Fibers, each of which is connected to a single Purkinje Fiber that integrates into an orthogonal layer of Parallel Fibers (composed of granule cells) through many synapses. Nearly all post-mortem examinations of ASD brains have identified differences in the cerebellum compared to controls, and the most consistent observations are the loss of Purkinje Fiber cells, overall cerebellar enlargement early in development, and reduction in size by adulthood. Functional differences of the cerebellum among ASD individuals are also widely reported. Although the inventors identify SV in all types of glutamate receptors and accessory proteins, the frequency of SV and the subunits affected strongly implicate the cerebellum in ASD. The inventors summarize each of the categories below.

### AMPAR—α-amino-3-hydroxy-5-methyl-4-isoxazolepropionic Acid Receptor

The majority of fast excitatory synaptic transmission in the mammalian central nervous systems is mediated by AMPA receptors that are heterodimers of one of the four subunit types (GRIA1-4). These receptors are also important for NMDA-modulated plasticity and as with other glutamate receptors, splice variants and different combinations of heterodimers produce a diversity of receptor types. AMPA typically modifies NMDA signaling by releasing voltage-dependent activity-blocks from extracellular Mg2+ to those receptor types. The GRIA2 subunit is unusual in that it undergoes RNA-editing, which directly affects the permeability of the channel pore itself and is the major form found in the adult brain. The majority of heterodimers of these receptors are composed of GRIA1 and 2 but GRIA4 is expressed highly in the developing neonatal brain and in the adult it is mainly found in the cerebellum as a homodimer in Bergmann's Glia (see GluD below) or interneurons. Deletion of the GRIA4 subtypes in these cells in young mice results in the disruptions between granule cells of the Parallel fiber layer and Purkinje cells.

Overall, the Inventors find that ASD cases have SVs in several GRIA subunits. As with all glutamate receptors, AMPAR have numerous accessory subunits that participate in presentation and signaling that include the stargazing family of proteins (CACNG1-8), the SHISA family of proteins, as well as IL1RAP1L, GRIP1 and GRIP2, and the tyrosine phosphatase PTPRD that binds to IL1RAPL1. Several of these have been associated with ASD in other work and display ASD-SV. Just under 15% of cases display an SV in CACNG2, which results in loss of excitatory transmission between mossy fibers and granule cells of the Parallel Fibers when deleted.

### NMDAR—N-methyl-D-aspartate Receptor

At most synapses, NMDA and AMPA are expressed at postsynaptic membranes and are co-activated by glutamate secreted from the presynaptic terminal. As with the other glutamate receptors, NMDA exists as multimers of different subunits, although all contain at least one GRIN1 subunit and usually GRIN2. In the instant analysis, many ASD cases carry an ASD-SV in at least one NMDA subunit as well as several supporting proteins for NMDA function. The majority of individuals harbor an SV in the KALRN gene, which is necessary for NMDA-dependent plasticity. The inventors did not detect an ASD-SV in the obligatory GRIN1 subunit, which may indicate strong purifying selection for proper function. The two subunits demonstrating the highest levels of ASD-SV (GRIN3A and GRIN2B), as with other SV-containing glutamate receptor subunits discussed here, are important for early postnatal development. Nearly ⅓ of individuals carry ASD-SV in GRIN3A, which alters NMDA signaling in a dominant negative manner when present. As GRIA4, GRIN3A is specific to and important for early brain development, which includes expression in astrocytes (e.g., Bergmann's glia). Finally, physical activity regulates expression of GRIN2B in cerebellum granule cells (Parallel Fibers).

### KAR—Kainate Receptor

KAR are unlike the other glutamate receptors in that they tend to modulate or regulate the synaptic activity of the other types and regulate neurotransmitter release. They are also necessary for a unique NMDA-independent form of plasticity in the hippocampus, an area that shows decreased activity in ASD and is linked to short term memory. Loss of function mutations in the GRIK2 subunit cause severe intellectual disability and appear to be responsible for mood disorders. KARs differ from NMDAR and AMPAR in that they can be present at both pre- and postsynaptic membranes. KAR have been shown to modulate synaptic transmission at mossy fiber-CA3 pyramidal cells, which feed directly to Purkinje cells in the cerebellum (GluD below). Many ASD cases carry an ASD-SV in at least one GRIK subunit of KARs with the majority occurring in GRIK2, a gene that has been associated with ASD in several other studies.

The most frequent ASD-SV site overlaps and is identified by the SNP rs2051449. This site resides 600 base pairs from a ChIP-Seq site for PCBP2, SRSF9, and HNRNPK, all of which participate in RNA-splicing. It is therefore likely that this ASD-SV disrupts proper splicing of the adjacent exon 12 of the gene. This likely results in the loss of exon 12, directly affecting the glutamate binding pocket. It is possible that the exon-depleted form of KAR assembles but does not signal, producing a dominant negative phenotype.

### GluD—Glutamate Receptor Delta

GluD receptors are an important component of the neurobiology of the cerebellum. There are two GluDs (GLUD1 and GLUD2 proteins encoded by GRID1 and GRID2 genes, respectively). GluD2 binds serine as well as a family of proteins called cerebellins (Cblns), which are secreted from granule cells onto Purkinje Fiber cells with the assistance of the Bergmann's Glia. The highly organized network of the cerebellum is disrupted in GRID2 knockout mice in several ways; rather than a single Climbing Fiber cell connecting to a single Purkinje Fiber cell, Climbing Cells connect to numerous Purkinje Cells and granule cells that comprise the Parallel Fibers in the orthogonal layer. It appears that these connections are meant to be pruned during brain development and the loss of GRID2 prevents this. In addition, AMPA receptors are expressed at much higher levels in GRID2 knockout mice than wildtype mice, suggesting that a normal function of GRID2 is to suppress AMPA expression. Unlike the other four glutamate receptors, GluDs do not directly bind glutamate. Most ASD individuals carry an ASD-SV in the GRID2 gene.

mGLURs—Metabotropic Glutamate Receptors

Unlike the other glutamate receptors, metabotropic glutamate receptors (mGLURs) are G-protein coupled receptors (GPCRs) that signal through a traditional intracellular cascade upon binding ligand instead of acting as an ionic channel as the other receptors do. mGLURs also exist as dimers rather than tetramers as most iGLURs. The eight known mGLURs are divided into three groups based on intracellular signaling and biological effect. Group 1 (GRM1 and GRM5) act to release intracellular calcium stores for propagation of signal whereas those in Groups 2 (GRM2 and GRM3) and Group 3 (GRMs 4,7, and 8) act through adenylate cyclase. These latter two groups also inhibit the release of the inhibitory neurotransmitter GABA (gamma-aminobutyric acid). More than half of cases harbor an SV in one of the Group I mGluRs, with the highest in GRM5. As with many of the other ASD-associated glutamate receptor subunits in this study, GRM5 is expressed early in development in Purkinje Fibers and declines into adulthood. GRM5 has been shown to immunoprecipitate and function with GluD1 (see GluD above), which results in altered AMPA expression. GRM1 and GRM5 also interact with NMDA receptors via DLG4, SHANK, and HOMER proteins, which have been implicated in ASD and function as associated proteins with GluDs. Finally, GRM5 has been shown to be a necessary component of AMPA/NMDA-mediated phosphorylation of moesin for dendritic spine development and axon guidance.

### Axon Guidance

The development of complex neural circuits requires the migration of axons over long distances to make the appropriate connections to their target cells. This process requires an axon guidance “cone” at the tip, which senses attractant or repulsive cues secreted by astrocytes and other cells that lie along the path. The axons turn based on the combination of the molecule secreted and the receptor(s) being expressed at the tip of the cone. Upon passing a secreting sentinel cell, the receptors at the tip are degraded and replaced with new receptors that will sense the next decision point in the pathway. Often the axon will make contacts with the cell it passes via contactin and contactin-associated proteins (CNTNs and CNTNAPs) that, as mentioned above, are part of the NCAM-associated SVs.

The majority of the axon-guidance related genes harboring ASD-SV are either the receptors expressed at the cone of the migrating axon or their partner ligand that is secreted by the cells at the choice point. The two most affected pairs are the Netrin/DCC and the ROBO1/SLIT1 genes followed by NRP1 and the Semaphorins. The largest group of axon guidance genes affected are the Ephrin receptors, which are heavily involved in the development of the superior colliculus, notably knockout mice of EPHA8 fail to develop proper connections within this structure (OMIM #176945). The superior colliculus functions to initiate behavioral responses to visual cues in the external world.

### Example 5

Detection of SVs is challenging, even when applying a combination of the most recent sequencing technology and variant calling algorithms, but important since SVs can have profound effects on complex traits. The instant NMI approach using SNP array data is rapid, inexpensive, flexible, and is able to identify complex and difficult to detect SVs, such as mobile element insertions, because the NMI pattern that reveals them is based directly on the binding of a 50 bp probe (i.e., local genomic variation) rather than probability-based mapping algorithms employed for long- and short-read sequencing data. Starting from a family-based pedigree population with a common phenotype of interest (e.g., a disease), the NMI workflow produces a set of high frequency SVs specific to that population (relative to the general population), and therefore potentially causative of their common phenotype.

Here, the inventors demonstrated the efficacy of the approach using a population of ASD parent-child trios as a case study. ASD is highly investigated, yet large scale GWAS tends to explain only a small proportion of the high heritability. The instant NMI workflow shows that the missing heritability may not be due to pleiotropy, somatic mutations or rare variants, as is often assumed, but instead may reside in previously undetected SVs that are revealed via pedigree datasets when NMI loci are retained rather than discarded. The set of high frequency ASD-specific SVs that were detected with the instant NMI approach provides an abundance of material for follow-up work. It is possible that some of these SVs only appear to be ASD-specific because they have not been discovered yet in the general population due to sequencing/genotyping limitations. However, the inventors were able to show that, in addition to many novel SVs, the set of ASD-specific SVs contains large proportions of SVs already present in databases such as AutDB. Furthermore, the genes harboring these ASD-specific SVs are significantly enriched for known ASD risk genes, and for highly relevant biological processes. Finally, by applying the workflow to both a discovery population (MIAMI) and an independent validation population (AGPC), the inventors were able to show that these ASD-specific SVs are reproducible and therefore provide new candidates for investigation. Critically, this resource has great potential to illuminate the genomic basis of ASD in greater detail than before because, in contrast to SFARI and AutDB which are comprised of rare risk genes, here the inventors generate a database of high-resolution loci that appear at high frequency amongst ASD cases. Thus, the NMI workflow can provide new insights into diseases, even from older datasets such as those used here.

As a demonstration, the inventors performed a mechanistic deep dive of a novel ASD-specific SV detected in the GRIK2 gene at high frequency. The inventors were able to use supporting RNA-seq data from ASD cases independent of the instant discovery population to show that GRIK2 exon 12 is lost at the location of this SV, likely causing significantly disrupted glutamate signaling. The inventors were also able to generate other highly specific hypotheses to test, e.g., ASD results from SVs in genes that regulate dendritic spine formation of Purkinje Fibers during early development of the cerebellum. The inventors also report a significant association of a variant in a regulatory site for the ACMSD gene with non-verbal ASD cases. This discovery implicates the kynurenine pathway in the disorder, which lies at the nexus of numerous ASD-associated traits including neuroinflammation, sleep disorder, gastrointestinal abnormalities, and altered circadian rhythms, as well as supports the major involvement of glutamate signaling imbalance in ASD. The ability to include SVs in these analyses has identified a previously unrecognized pathway for possible pharmaceutical intervention.

Beyond ASD, it is likely that such undetected SVs are the key “missing heritability” needed to explain many other diseases and phenotypes. Amyotrophic lateral sclerosis (ALS), like ASD, is a heterogeneous disorder with an estimated heritability of 65%, and yet large-scale genomic analyses have only identified markers that explain about 10% of cases. Recently, it was discovered SVs caused by expansion of repetitive microsatellite elements in two genes (C9orf27 and ATXN2) to cause some cases of ALS. Likewise, the heritability of late onset Alzheimer's disease (LOAD) is at least 60%, and although the epsilon 4 allele of ApoE accounts for roughly a quarter of that heritability, it does not fully explain age of onset or the remaining cases. However, an SV in the neighboring gene TOMM40, which likely represents a hotspot for transposon activity, increases the LOAD risk odds ratio by 4-fold compared to the ApoE e4 allele alone. The inventors predict this approach will rapidly advance the knowledge of the genetic basis of many health conditions of societal importance, as well improve the discovery of key markers for genomic breeding in agricultural applications.

What is claimed is:

1. A method of identifying at least one structural variation in a genome, the method comprising:
assembling single nucleotide polymorphism (SNP) data from parents and their offspring;
analyzing the SNP data for a plurality of non-Mendelian inheritance (NMI) patterns, wherein each NMI in the plurality of NMI patterns is a potential structural variation;
scoring the NMIs to identify large structural variations, wherein a run of at least three SNPs with NMI indicates a large structural variation;
removing SNPs that demonstrate NMI in the offspring but that overlap with at least one known existing variation;
identifying conserved regions of the genome to filter regions that should be conserved but include a structural variation;
identifying biologically important structural variations; and
classifying the identified biologically important structural variations using a machine learning algorithm.

2. The method of claim 1, wherein the machine learning algorithm is a neural network.

3. The method of claim 1, wherein the machine learning algorithm is an iterative Random Forest (iRF).

4. The method of claim 1, further comprising determining the frequency an NMI and comparing the frequency of NMIs at the corresponding genomic location in a population, and determining that the NMI indicates a structural variation if the frequency of the NMI is higher than that of the corresponding genomic region in the population.

5. The method of claim 1, wherein the biologically important structural variations are selected from structural variations that reside in a gene in which less than 5% of normal individuals have a known structural variation; and there is a run of at least four SNPs with NMI in a row.

6. The method of claim 1, wherein identifying conserved regions of the genome is performed by a custom correlation coefficient (CCC) analysis.

7. The method of claim 1, further comprising assigning a probability score for having a run of NMI greater than 4.

8. The method of claim 1, comprising removing NMI attributable to high levels of masked repetitive elements.

9. The method of claim 1, comprising identifying pinpoint locations of the structural variations and identifying pinpoint locations of conserved blocs of genetic information.

10. The method of claim 9, comprising using the locations of the structural variations and the locations of the conserved blocs of genetic information to identify locations of rare structural variations in genes that have conserved blocs of genetic information.

11. A computer-implemented method of training a machine learning algorithm for identifying at least one structural variation in a genome, the method comprising
training the machine learning algorithm using a training set, wherein the training set is created by:
assembling single nucleotide polymorphism (SNP) data from parents and their offspring;
analyzing the SNP data for a plurality of non-Mendelian inheritance patterns (NMI), wherein each NMI is a potential structural variation;
scoring the NMIs to identify large structural variations, wherein presence of at least three neighboring SNPs that demonstrate NMI in the offspring indicates a large structural variation;
removing SNPs that demonstrate NMI in the offspring but that overlap with at least one known existing variation;
identifying conserved regions of the genome to filter regions that should be conserved but include a structural variation; and
identifying potentially biologically important structural variations.

12. The computer-implemented method of claim 11, wherein the machine learning algorithm is a neural network.

13. The computer-implemented method of claim 11, wherein the machine learning algorithm is an iterative Random Forest.

14. A processor programmed to perform:
assembling single nucleotide polymorphism (SNP) data from parents and their offspring;
analyzing the SNP data for a plurality of non-Mendelian inheritance (NMI) patterns, wherein each NMI in the plurality of NMI patterns is a potential structural variation;
scoring the NMIs to identify large structural variations, wherein the presence of at least three neighboring SNPs that demonstrate NMI in the offspring indicates a large structural variation;
removing SNPs that demonstrate NMI in the offspring but that overlap with at least one known existing variation;
identifying conserved regions of the genome to filter regions that should be conserved but include a structural variation;
identifying biologically important structural variations; and
classifying the identified biologically important structural variations using a machine learning algorithm.

15. The processor of claim 14, wherein the machine learning algorithm is a neural network.

16. The processor of claim 14, wherein the machine learning algorithm is an iterative Random Forest.

17. The processor of claim 14, further comprising determining the frequency an NMI and comparing the frequency of NMIs at the corresponding genomic location in a population, and determining that the NMI indicates a structural variation if the frequency of the NMI is higher than that of the corresponding genomic region in the population.

18. The processor of claim 14, wherein the biologically important structural variations are selected from structural variations that reside in a gene in which less than 5% of normal individuals have a known structural variation; and there is a run of at least four SNPs with NMI in a row.

19. The processor of claim 14, wherein identifying conserved regions of the genome is performed by a custom correlation coefficient (CCC) analysis.

20. The processor of claim 14, further comprising assigning a probability on having a run of NMI greater than 4.

21. The processor of claim 14, comprising removing NMI attributable to high levels of masked repetitive elements.

22. The processor of claim 14, comprising identifying pinpoint locations of the structural variations and identifying pinpoint locations of conserved blocs of genetic information.

23. The processor of claim 22, comprising using the locations of the structural variations and the locations of the conserved blocs of genetic information to identify locations of rare structural variations in genes that have conserved blocs of genetic information.

24. A computer-readable storage device, comprising instructions to perform:
assembling single nucleotide polymorphism (SNP) data from parents and their offspring;
analyzing the SNP data for a plurality of non-Mendelian inheritance (NMI) patterns, wherein each NMI in the plurality of NMI patterns is a potential structural variation;
scoring the NMIs to identify large structural variations, wherein the presence of at least three neighboring SNPs that demonstrate NMI in the offspring indicates a large structural variation;
removing SNPs that demonstrate NMI in the offspring but that overlap with at least one known existing variation;
identifying conserved regions of the genome to filter regions that should be conserved but include a structural variation;
identifying biologically important structural variations; and
classifying the identified biologically important structural variations using a machine learning algorithm.

25. The computer-readable storage device of claim 24, wherein the machine learning algorithm is a neural network.

26. The computer-readable storage device of claim 24, wherein the machine learning algorithm is an iterative Random Forest.

27. The computer-readable storage device of claim 24, further comprising determining the frequency an NMI and comparing the frequency of NMIs at the corresponding genomic location in a population, and determining that the NMI indicates a structural variation if the frequency of the NMI is higher than that of the corresponding genomic region in the population.

28. The computer-readable storage device of claim 24, wherein the biologically important structural variations are selected from structural variations that reside in a gene in which less than 5% of normal individuals have a known structural variation; and there is a run of at least four SNPs with NMI in a row.

29. The computer-readable storage device of claim 24, wherein identifying conserved regions of the genome is performed by a custom correlation coefficient (CCC) analysis.

30. The computer-readable storage device of claim 24, further comprising assigning a probability on having a run of NMI and maintaining SNP's with a run of NMI greater than 4.

31. The computer-readable storage device of claim 24, comprising removing NMI attributable to high levels of masked repetitive elements.

32. The computer-readable storage device of claim 24, comprising identifying pinpoint locations of the structural variations and identifying pinpoint locations of conserved blocs of genetic information.

33. The computer-readable storage device of claim 32, comprising using the locations of the structural variations and the locations of the conserved blocs of genetic information to identify locations of rare structural variations in genes that have conserved blocs of genetic information.

34. A method comprising:
obtaining a biological sample from a subject,
detecting in the biological sample whether at least one gene or genomic region selected from Table 1 or Table 2 has a structural variation; and
determining that the subject is at risk of Autism Spectrum Disorder if the at least one gene or genomic region has a structural variation.

35. The method of claim 1, wherein the at least one gene further comprises GRIK2.

36. The method of claim 1, wherein the at least one gene further comprises ACMSD.
